(19) United States
(12) Patent Application Publication
(10) Pub. No.: US 2024/0000704 A1
(43) Pub. Date: Jan. 4, 2024

(54) METHODS OF USING UMBILICAL CORD PRODUCTS FOR TREATMENT OF OCULAR SURFACE DISORDERS

(71) Applicant: TissueTech, Inc., Miami, FL (US)

(72) Inventors: Scheffer TSENG, Pinecrest, FL (US); Sean TIGHE, Fort Lauderdale, FL (US); Ek Kia TAN, Miami, FL (US); Jason Eric VEVODA, Happy Valley, OR (US)

(21) Appl. No.: 17/914,691

(22) PCT Filed: Apr. 2, 2021
(86) PCT No.: PCT/US2021/025642

§ 371 Date: Sep. 26, 2022

Related U.S. Application Data
(60) Provisional application No. 63/004,987, filed on Apr. 3, 2020.

(51) Int. Cl. A61K 9/00; A61K 35/51; A61P 27/02; A61K 35/50; A61K 45/06

(52) U.S. Cl. CPC A61K 9/0051; A61K 35/51; A61P 27/02; A61K 35/50; A61K 45/06

(57) ABSTRACT

Disclosed herein are methods of treating an ocular surface disease, disorder, or wound comprising applying an umbilical cord sheet and/or a controlled-release formulation to a tissue or surrounding tissue of an eye of a subject, such as the inferior fornix, superior fornix, and/or cornea. Further disclosed herein are kits for treatment of an ocular surface disease, disorder, or wound comprising the umbilical cord sheet and/or controlled-release formulation and a device for delivering and applying the umbilical cord sheet and/or controlled-release formulation to an eye of a subject.

### CROSS-REFERENCE

This application claims the benefit of U.S. Provisional Patent Application No. 63/004,987 filed on Apr. 3, 2020, which is incorporated herein by reference in its entirety.

### SUMMARY

Heavy chain-hyaluronan/pentraxin 3 (HC-HA/PTX3) complex, which is found in fetal support tissues such as amniotic membrane and umbilical cord, has anti-inflammatory, anti-scarring and wound healing properties.

Disclosed herein is a method of treating an ocular surface disease, disorder, and/or wound, the method comprising placing an umbilical cord sheet into an inferior fornix, superior fornix, or both of an eye of a subject having an ocular surface disease, disorder, and/or wound. In some embodiments, the method comprises placing an umbilical cord sheet into an inferior fornix only of an eye of a subject having an ocular surface disease, disorder, and/or wound. In some embodiments, the method comprises retracting an upper eyelid, lower eyelid, or both of the subject to expose the inferior fornix, superior fornix, or both prior to placing the umbilical cord sheet. In some embodiments, the method comprises patching or taping the eye closed. In some embodiments, the method comprises administering to the eye, a fetal support tissue product selected from the group consisting of: placental amniotic membrane, placenta, chorion, umbilical cord, umbilical cord amniotic membrane, and a combination thereof. In some embodiments, the method comprises administering to the eye, a therapeutic agent selected from the group consisting of: a steroid, an antibacterial agent, an anti-inflammatory agent, a dry eye treatment agent, an anti-fungal agent, an anti-viral agent, an anti-glaucoma agent, and any combination thereof. In some embodiments, the therapeutic agent or the fetal support tissue product is administered to the inferior fornix, superior fornix, or both of the eye, the superior fornix, a corneal surface of the eye, and/or tissue surrounding the eye. In some embodiments, the therapeutic agent or the fetal support tissue product is administered prior to placing the umbilical cord sheet. In some embodiments, the umbilical cord sheet comprises umbilical cord amniotic membrane and Wharton's jelly. In some embodiments, the umbilical cord sheet is from frozen or previously-frozen umbilical cord. In some embodiments, the umbilical cord sheet does not comprise a cell with metabolic activity, or is substantially free of cells with metabolic activity. In some embodiments, the umbilical cord sheet comprises cells all, substantially all, or most of which are dead. In some embodiments, the umbilical cord sheet is substantially free of red blood cells. In some embodiments, the umbilical cord sheet is cryopreserved, terminally sterilized, or both. In some embodiments, the umbilical cord sheet is substantially flat. In some embodiments, the umbilical cord sheet is semi-circular. In some embodiments, the umbilical cord sheet is circular. In some embodiments, the umbilical cord sheet is rectangular. In some embodiments, the umbilical cord sheet is about 1.0-2.0 cm by about 0.1-0.5 cm. In some embodiments, the umbilical cord sheet is about 1.5 cm by about 0.3 cm. In some embodiments, the umbilical cord sheet is substantially free of a vein or an artery. In some embodiments, the umbilical cord sheet is hydrated. In some embodiments, the umbilical cord sheet promotes nerve regeneration in the eye of the subject. In some embodiments, the umbilical cord sheet is anti-inflammatory, anti-scarring, anti-angiogenic, anti-adhesion, pain-reducing, promotes wound healing, or a combination thereof. In some embodiments, the ocular disease or disorder is selected from the group consisting of dry eye disease, recurrent corneal erosion (RCE), corneal ulcers, herpes simplex keratitis (HSK), recalcitrant punctate keratitis, allergic conjunctivitis, pterygium, infectious diseases, and inflammatory diseases. In some embodiments, the wound is a surgical wound. In some embodiments, the wound is from photorefractive keratectomy (PRK), LASIK, corneal collagen cross-linking (CXL), corneal transplantation, cataract surgery, retinal surgery, and/or a glaucoma drainage device or filtering bleb. In some embodiments, the wound is from an injury, burn, laceration, incision, or abrasion.

In some embodiments, disclosed herein is a kit for treatment of an ocular surface disease, disorder, or wound in a subject in need thereof, the kit comprising a) an umbilical cord sheet; and b) a device for placing the umbilical cord sheet onto a corneal surface, into an inferior fornix, a superior fornix, or onto a tissue surrounding an eye of the subject, the device comprising: i) a storage unit configured to contain the umbilical cord sheet in a solution; and ii) a channel operatively coupled to the storage unit, the channel configured such that the umbilical cord sheet passes through the channel from the storage unit and through an opening in the channel, the opening configured to deliver the umbilical cord sheet onto the corneal surface, inferior fornix, a superior fornix, or tissue surrounding of the eye. In some embodiments, the device further comprises an application member operatively coupled to the channel, wherein the application member is configured to push the umbilical cord sheet through the opening. In some embodiments, the channel comprises a porous barrier comprising one or more pores, wherein at least one pore of the one or more pores is sized to inhibit the umbilical cord sheet from passing therethrough. In some embodiments, the umbilical cord sheet does not comprise a cell with metabolic activity, or is substantially free of cells with metabolic activity. In some embodiments, the umbilical cord sheet is substantially-flattened. In some embodiments, the umbilical cord sheet is semi-circular. In some embodiments, the umbilical cord sheet is circular. In some embodiments, the umbilical cord sheet is rectangular. In some embodiments, the umbilical cord sheet comprises umbilical cord amniotic membrane and Wharton's jelly. In some embodiments, the umbilical cord sheet is from frozen or previously-frozen umbilical cord. In some embodiments, the umbilical cord sheet comprises cells substantially all of which are dead. In some embodiments, the umbilical cord sheet is hydrated. In some embodiments, the umbilical cord sheet promotes nerve regeneration in the eye of the subject. In some embodiments, the umbilical cord sheet is anti-inflammatory, anti-scarring, anti-angiogenic, anti-adhesion, pain-reducing, and/or promotes wound healing when contacted with an exogenous living cell. In some embodiments, the kit further comprises a therapeutic agent selected from the group consisting of: a steroid, an antibacterial agent, an anti-inflammatory agent, a dry eye treatment agent, an anti-fungal agent, an anti-viral agent, an anti-glaucoma agent, and any combination thereof. In some embodiments, the kit further comprises a fetal support tissue product selected from the group consisting of: placental amniotic membrane, placenta, chorion, umbilical cord, umbilical cord amniotic membrane, and a combination thereof.

In some embodiments, disclosed herein is a method of treating an ocular surface disease, disorder, or wound in a subject in need thereof, the method comprising administering to an inferior fornix, a superior fornix, or a corneal surface of an eye of the subject a composition comprising isolated HC-HA/PTX3 and an excipient for controlled-release of the HC-HA/PTX3. In some embodiments, the excipient for controlled-release comprises a biodegradable polymer. In some embodiments, the excipient for controlled-release comprises collagen, cellulose, chitosan, PEG, poly(N-isopropylacrylamide), poly(lactic) acid, poly(lactic-co-glycolic) acid, or a combination thereof. In some embodiments, the composition conforms to the inferior fornix, the superior fornix, or the corneal surface. In some embodiments, the composition is a solid or semi-solid. In some embodiments, the composition comprises an opening. In some embodiments, the composition is semi-circular. In some embodiments, the composition is circular. In some embodiments, the composition is rectangular. In some embodiments, the composition is about 1.0-2.0 cm by about 0.1-0.5 cm. In some embodiments, the composition is about 1.5 cm by about 0.3 cm. In some embodiments, the method further comprises administering to the eye, a therapeutic agent selected from the group consisting of: a steroid, an antibacterial agent, an anti-inflammatory agent, a dry eye treatment agent, an anti-fungal agent, an anti-viral agent, an anti-glaucoma agent, and any combination thereof. In some embodiments, the method further comprises administering to the eye a fetal support tissue product selected from the group consisting of: placental amniotic membrane, placenta, chorion, umbilical cord, umbilical cord amniotic membrane, and a combination thereof. In some embodiments, the therapeutic agent or the fetal support tissue is administered to the inferior fornix of the eye, the superior fornix, or a corneal surface of the eye, and/or tissue surrounding the eye. In some embodiments, the therapeutic agent or the fetal support tissue product is administered prior to the composition.

In some embodiments, disclosed herein is a composition comprising an isolated HC-HA/PTX3 complex and an excipient for controlled-release, wherein the composition conforms to a corneal surface or fits in an inferior fornix or a superior fornix of a human eye. In some embodiments, the composition is anti-inflammatory, anti-scarring, anti-angiogenic, anti-adhesion, pain-reducing, promotes wound healing, or a combination thereof. In some embodiments, the excipient for controlled-release comprises a biodegradable polymer. In some embodiments, the excipient for controlled-release comprises collagen, cellulose, chitosan, PEG, poly(N-isopropylacrylamide), poly(lactic) acid, poly(lactic-co-glycolic) acid, or a combination thereof. In some embodiments, the composition is transparent, or translucent. In some embodiments, the composition is a solid or a semi-solid. In some embodiments, the composition is flat or substantially flat. In some embodiments, the composition comprises an opening. In some embodiments, the composition is semi-circular. In some embodiments, the composition is circular. In some embodiments, the composition is rectangular. In some embodiments, the composition is about 1.0-2.0 cm by about 0.1-0.5 cm. In some embodiments, the composition is about 1.5 cm by about 0.3 cm.

In some embodiments, disclosed herein is a kit for treatment of an ocular surface disease, disorder, or wound in a subject, the kit comprising: a) a composition comprising isolated HC-HA/PTX3 and an excipient for controlled-release of HC-HA/PTX3; and b) a device for placing the composition onto a corneal surface, into an inferior fornix, a superior fornix, or onto a tissue surrounding an eye of the subject, the device comprising: i) a storage unit configured to contain the composition in a solution; and ii) a channel operatively coupled to the storage unit, the channel configured such that the composition passes through the channel from the storage unit and through an opening in the channel, the channel configured to deliver the composition onto the corneal surface, the inferior fornix, the superior fornix, or tissue surrounding the eye. In some embodiments, the device further comprises an application member operatively coupled to the channel, wherein the application member is configured to push the umbilical cord sheet through the opening so as to be placed onto the tissue and/or surrounding tissue of the eye. In some embodiments, the channel comprises a porous barrier comprising one or more pores, wherein at least one pore of the one or more pores is sized to inhibit the umbilical cord sheet from passing therethrough. In some embodiments, the composition is a solid or semi-solid. In some embodiments, the composition is semi-circular. In some embodiments, the composition is circular. In some embodiments, the composition is rectangular. In some embodiments, the composition promotes nerve regeneration in the eye of the subject. In some embodiments, the kit further comprises a therapeutic agent that is selected from the group consisting of: a steroid, an antibacterial agent, an anti-inflammatory agent, an ophthalmic drop, dry eye treatment agent; anti-fungal agent, anti-viral agent, or any combination. In some embodiments, the kit further comprises a fetal support tissue product selected from the group consisting of: placental amniotic membrane, placenta, chorion, umbilical cord, umbilical cord amniotic membrane or a combination thereof.

Disclosed herein is a method of treating an ocular surface disease, disorder, and/or wound, comprising: placing an umbilical cord sheet of about 0.75-2.5 cm by about 0.05-0.75 cm in an inferior fornix, superior fornix, or both of an eye of a subject having an ocular surface disease, disorder, and/or wound. In some embodiments, the method comprises retracting an upper eyelid, lower eyelid, or both of the subject to expose the inferior fornix prior to placing the umbilical cord sheet. In some embodiments, the method comprises patching or taping the eye closed. In some embodiments, the method comprises administering to the eye a fetal support tissue product selected from the group consisting of: placental amniotic membrane, placenta, chorion, umbilical cord, umbilical cord amniotic membrane, and a combination thereof. In some embodiments, the method comprises administering to the eye a therapeutic agent selected from the group consisting of: a steroid, an antibacterial agent, an anti-inflammatory agent, a dry eye treatment agent, an anti-fungal agent, an anti-viral agent, an anti-glaucoma agent, and any combination thereof. In some embodiments, the therapeutic agent or the fetal support tissue product is administered to the inferior fornix of the eye, a corneal surface of the eye, and/or tissue surrounding the eye. In some embodiments, the therapeutic agent or the fetal support tissue product is administered prior to placing the umbilical cord sheet. In some embodiments, the umbilical cord sheet comprises umbilical cord amniotic membrane and Wharton's jelly. In some embodiments, the umbilical cord sheet is from frozen or previously-frozen umbilical cord. In some embodiments, the umbilical cord sheet does not comprise a cell with metabolic activity, or is substantially free of cells with metabolic activity. In some embodiments, the umbilical cord sheet comprises cells all, substantially all, or most of which are dead. In some embodiments, the umbilical cord sheet is substantially free of living cells. In some embodiments, the umbilical cord sheet is substantially free of red blood cells. In some embodiments, the umbilical cord sheet is substantially free of a vein or an artery. In some embodiments, the umbilical cord sheet is cryopreserved, terminally sterilized, or both. In some embodiments, the umbilical cord sheet is substantially flat. In some embodiments, the umbilical cord sheet is semi-circular. In some embodiments, the umbilical cord sheet is circular. In some embodiments, the umbilical cord sheet is rectangular. In some embodiments, the umbilical cord sheet is about 1.0-2.0 cm by about 0.1-0.5 cm. In some embodiments, the umbilical cord sheet is about 1.5 cm by about 0.3 cm. In some embodiments, the umbilical cord sheet is hydrated. In some embodiments, the umbilical cord sheet promotes nerve regeneration in the eye of the subject. In some embodiments, the umbilical cord sheet is anti-inflammatory, anti-scarring, anti-angiogenic, anti-adhesion, pain-reducing, promotes wound healing, or a combination thereof. In some embodiments, the ocular disease or disorder is selected from the group consisting of dry eye disease, recurrent corneal erosion (RCE), corneal ulcers, herpes simplex keratitis (HSK), recalcitrant punctate keratitis, allergic conjunctivitis, pterygium, infectious diseases, and inflammatory diseases. In some embodiments, the wound is a surgical wound. In some embodiments, the wound is from photorefractive keratectomy (PRK), LASIK, corneal collagen cross-linking (CXL), corneal transplantation, cataract surgery, retinal surgery, or a glaucoma drainage device or filtering bleb. In some embodiments, the wound is from an injury, burn, laceration, incision, or abrasion.

Disclosed herein is a method of treating an ocular surface disease, disorder, and/or wound, comprising: placing an umbilical cord sheet in an inferior fornix only of an eye of a subject having an ocular surface disease, disorder, and/or wound. In some embodiments, the method comprises retracting an upper eyelid, lower eyelid, or both of the subject to expose the inferior fornix prior to placing the umbilical cord sheet. In some embodiments, the method comprises patching or taping the eye closed. In some embodiments, the method comprises administering to the eye a fetal support tissue product selected from the group consisting of: placental amniotic membrane, placenta, chorion, umbilical cord, umbilical cord amniotic membrane, and a combination thereof. In some embodiments, the method comprises administering to the eye a therapeutic agent selected from the group consisting of: a steroid, an antibacterial agent, an anti-inflammatory agent, a dry eye treatment agent, an anti-fungal agent, an anti-viral agent, an anti-glaucoma agent, and any combination thereof. In some embodiments, the umbilical cord sheet is about 1.5 cm by about 0.3 cm.

Disclosed herein is a method of treating recurrent corneal erosion (RCE), corneal ulcers, herpes simplex keratitis (HSK), or OSD with recalcitrant punctate keratitis, comprising: placing an umbilical cord sheet in an inferior fornix or a superior fornix of an eye of a subject having recurrent corneal erosion (RCE), corneal ulcers, herpes simplex keratitis (HSK), or OSD with recalcitrant punctate keratitis. In some embodiments, the method comprises retracting an upper eyelid, lower eyelid, or both of the subject to expose the inferior fornix or the superior fornix prior to placing the umbilical cord sheet. In some embodiments, the method comprises patching or taping the eye closed. In some embodiments, the method comprises administering to the eye a fetal support tissue product selected from the group consisting of: placental amniotic membrane, placenta, chorion, umbilical cord, umbilical cord amniotic membrane, and a combination thereof. In some embodiments, the method comprises administering to the eye a therapeutic agent selected from the group consisting of: a steroid, an antibacterial agent, an anti-inflammatory agent, a dry eye treatment agent, an anti-fungal agent, an anti-viral agent, an anti-glaucoma agent, and any combination thereof. In some embodiments, the therapeutic agent or the fetal support tissue product is administered to the inferior fornix or the superior fornix of the eye, a corneal surface of the eye, and/or tissue surrounding the eye. In some embodiments, the therapeutic agent or the fetal support tissue product is administered prior to placing the umbilical cord sheet. In some embodiments, the umbilical cord sheet is about 1.0-2.0 cm by about 0.1-0.5 cm. In some embodiments, the umbilical cord sheet is about 1.5 cm by about 0.3 cm.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a non-limiting example of a device for delivering and placing an umbilical cord sheet and/or controlled-released formulation in an interior fornix, corneal surface, or tissue surrounding an eye of a subject.

FIG. 2A depicts a non-limiting example of an umbilical cord sheet and/or controlled-released formulation passing through a channel, downstream a porous barrier.

FIG. 2B depicts a non-limiting example of an application member configured to aid in delivering an umbilical cord sheet and/or controlled-released formulation through a channel opening.

FIG. 3A depicts a non-limiting example of a measurement of an umbilical cord tissue flattened on a frame backing paper, as part of a preparation process for an umbilical cord sheet.

FIG. 3B depicts a non-limiting example of an umbilical cord tissue cut longitudinally.

FIG. 3C depicts a non-limiting example of an umbilical cord tissue after a shorter cut, following the longitudinal cut.

FIG. 4 depicts a non-limiting example of an umbilical cord sheet being placed in the inferior fornix of a subject's eye.

FIG. 5A depicts a non-limiting example of an umbilical cord sheet placed in the inferior fornix, on the day of placement, the umbilical cord sheet displayed using fluorescence.

FIG. 5B depicts a non-limiting example of an umbilical cord sheet situated in the inferior fornix, one day post-placement, the umbilical cord sheet displayed using fluorescence.

FIG. 5C depicts a non-limiting example of an umbilical cord sheet situated in the inferior fornix, two days post-placement, the umbilical cord sheet displayed using fluorescence.

FIG. 6 depicts a packaging design for storage and placement of an umbilical cord sheet in an inferior fornix of an eye.

FIG. 7 depicts an alternative packaging design for storage and placement of an umbilical cord sheet in an inferior fornix of an eye.

FIG. 8 depicts a method of placing an umbilical cord sheet using the package of FIG. 7 in an inferior fornix of an eye.

FIG. 9 depicts yet another alternative packaging design for storage and placement of an umbilical cord sheet in an inferior fornix of an eye.

FIGS. 10A-10E depict survey data from patients who used the UC strips for mild/moderate or severe indications. Tolerance, ease of insertion, product retention, clinical benefits, patient satisfaction, and physician satisfaction were surveyed.

### DETAILED DESCRIPTION

Heavy chain-hyaluronan/pentraxin 3 (HC-HA/PTX3) complex, which is found in fetal support tissues such as amniotic membrane and umbilical cord, has anti-inflammatory, anti-scarring and wound healing properties. Provided herein are methods and systems for the controlled release of HC-HA/PTX3 into the tear film of an eye to promote wound healing, decrease inflammation, reduce scarring, reduce pain, or a combination thereof, in a subject having an ocular disease, disorder, or wound. In some embodiments, the methods and controlled release systems comprise a substantially flattened sheet, strip, or piece of umbilical cord that is placed in the inferior fornix, a superior fornix, the punctum, onto a corneal surface, or onto a tissue surrounding the eye of a subject thereby providing a controlled release of HC-HA/PTX3 into the tear film of the subject. In some embodiments, the methods and controlled release systems comprise a substantially flattened sheet, strip, or piece of umbilical cord that is placed in the inferior fornix only of the eye of a subject thereby providing a controlled release of HC-HA/PTX3 into the tear film of the subject.

In some embodiments, the HC-HA/PTX3 is released through a controlled-release formulation, comprising HC-HA/PTX3 complex and an excipient, which is placed in an inferior fornix, a superior fornix, the punctum, onto a corneal surface, or onto a tissue surrounding the eye. In some embodiments, the excipient enables a controlled release of the HC-HA/PTX3 in the inferior fornix, the superior fornix, the punctum, onto a corneal surface, or onto the tissue surrounding the eye. As used herein, controlled release refers to a sustained release of HC-HA/PTX3 into the inferior fornix, into the superior fornix, onto the punctum, onto a corneal surface, or onto tissue surrounding an eye of a subject. The sustained release can be a uniform rate of release or varying rate of release of HC-HA/PTX3 over a period of time. As used herein, the terms applied and placed are interchangeable.

### Preparation of Umbilical Cord Sheet

In some embodiments, umbilical cord tissue is harvested from any suitable donor source (e.g., a hospital or tissue bank) to prepare for the umbilical cord sheet. In some embodiments, umbilical cord tissue is obtained from any mammal, such as a human, non-human primate, cow or pig.

All processing is done following Good Tissue Practices (GTP) to ensure that no contaminants are introduced into the umbilical cord sheet. In some embodiments, the harvested umbilical cord is tested for HIV-1, HIV-2, HTLV-1, hepatitis B and C, West Nile virus, cytomegalovirus, human transmissible spongiform encephalopathy (e.g., Creutzfeldt-Jakob disease) and/or treponema pallidum using FDA licensed screening test. Any indication that the tissue is contaminated with HIV-1, HIV-2, HTLV-1, hepatitis B and C, West Nile virus, or cytomegalovirus can result in the immediate quarantine and subsequent destruct of the tissue specimen.

In some embodiments, the donor's medical records are examined for risk factors for and clinical evidence of hepatitis B, hepatitis C, or HIV infection. Any indication that the donor has risk factors for, and/or clinical evidence of, infection with HIV-1, HIV-2, HTLV-1, hepatitis B and C, West Nile virus, cytomegalovirus, human transmissible spongiform encephalopathy (e.g., Creutzfeldt-Jakob disease) and/or treponema pallidum can result in the immediate quarantine and subsequent destruct of the tissue specimen.

In some embodiments, the umbilical cord is frozen. In some embodiments, the umbilical cord is not frozen. If the umbilical cord is not frozen, it can be processed as described below immediately.

In some embodiments, substantially all of the blood is removed from the umbilical cord. In some embodiments, substantially all of the blood is removed from the umbilical cord before the umbilical cord is frozen. In some embodiments, substantially all of the blood is removed from the arteries and veins of the umbilical cord. In some embodiments, blood is not removed from the UC. In some embodiments, blood is not removed from the umbilical cord before the umbilical cord is frozen. In some embodiments, the umbilical cord tissue is washed with buffer with agitation to remove excess blood and tissue. In some embodiments, washing with agitation reduces the wash time. In some embodiments, the umbilical cord tissue is contacted with a buffer to remove substantially all of the red blood cells. In some embodiments, the umbilical cord tissue is lyophilized, cryopreserved, pulverized, and/or terminally sterilized.

In some embodiments, the umbilical cord is washed with an hypertonic buffer or tissue culture media. In some embodiments, the umbilical cord is washed with an hypotonic buffer or tissue culture media. In some embodiments, the umbilical cord is washed with an isotonic buffer or tissue culture media. In some embodiments, the umbilical cord is washed with saline. In some embodiments, the umbilical cord is washed with PBS. In some embodiments, the umbilical cord is washed with PBS 1×. In some embodiments, the umbilical cord is washed with a TRIS-buffered saline. In some embodiments, the umbilical cord is washed with a HEPES-buffered saline. In some embodiments, the umbilical cord is washed with Ringer's solution. In some embodiments, the umbilical cord is washed with Hartmann's solution. In some embodiments, the umbilical cord is washed with EBSS. In some embodiments, the umbilical cord is washed with HBSS. In some embodiments, the umbilical cord is washed with Tyrode's Salt Solution. In some embodiments, the umbilical cord is washed with Gey's Balanced Salt Solution. In some embodiments, the umbilical cord is washed with Eagle's minimal essential medium (DMEM). In some embodiments, the umbilical cord is washed with Eagle's minimum essential medium (EMEM). In some embodiments, the umbilical cord is washed with Glasgow minimum essential medium (GMEM). In some embodiments, the umbilical cord is washed with Roswell Park Memorial Institute (RPMI) 1640 medium.

In some embodiments, a section of the umbilical cord is then cut longitudinally (e.g., using a scalpel or scissors). In some embodiments, the section of the umbilical cord is not cut into halves. In some embodiments, the section of the umbilical cord is cut into two halves.

In some embodiments, the cut umbilical cord tissue is optionally washed again with buffer to further remove excess blood and tissue.

In some embodiments, the umbilical cord is fastened onto a substrate (e.g., a styrofoam board) using any suitable method (e.g., it is fastened with needles or pins (e.g., T pins)). In some embodiments, the umbilical cord is stabilized with a substrate (e.g., absorbent towel cloth, drape). In some embodiments, the umbilical cord is oriented such that the inside face of the umbilical cord (e.g., the face comprising the Wharton's Jelly) is facing up while the outside face (i.e., the face comprising UCAM) is facing the substrate. Optionally, in some embodiments, one end of the umbilical cord is left free. If one end of the umbilical cord is left free, in some embodiments, the free end of the umbilical cord is held (e.g., with a clamp, hemostats or a set of forceps (e.g., wide serrated tip forceps)) while part or all of the Wharton's Jelly is removed. Alternatively, in some embodiments, both ends of the umbilical cord are left free.

If the umbilical cord does not lay flat against the substrate, in some embodiments, additional cuts are made in the Wharton's Jelly.

In some embodiments, part or all of the Wharton's Jelly is removed from the UCAM. The desired thickness of the sheet determines how much of the Wharton's Jelly is removed. In some embodiments, the Wharton's Jelly is peeled from the umbilical cord in layers (e.g., using a set of forceps, hemostats). In some embodiments, the Wharton's Jelly is cut away (e.g., shaved) from the umbilical cord in sections. In some embodiments, a rotoblator (i.e., a catheter attached to a drill with a diamond coated burr) is utilized to remove the Wharton's Jelly. In some embodiments, a liposuction machine is utilized to remove the Wharton's Jelly. In some embodiments, a liquid under high pressure is applied to remove the Wharton's Jelly. In some embodiments, a brush is utilized to remove the Wharton's Jelly (e.g., a mechanized brush rotating under high speed).

The umbilical cord comprises two arteries (the umbilical arteries) and one vein (the umbilical vein). In some embodiments, the vein and arteries are removed from the umbilical cord. In some embodiments, the vein and arteries are removed concurrently with the removal of some or all of the Wharton's Jelly by any of the below methods. In some embodiments, the vein and arteries are peeled (or pulled) from the umbilical cord (e.g., using a set of forceps). In some embodiments, the vein and arteries are cut away (e.g., shaved) from the umbilical cord in sections. In some embodiments, a rotoblator removes the vein and arteries. In some embodiments, a liposuction machine is utilized to remove the vein and arteries. In some embodiments, a vein stripper is utilized to remove the vein and arteries. In some embodiments, a liquid under high pressure removes the vein and arteries concurrently. In some embodiments, a brush removes the vein and arteries. In some embodiments, a surgical dermatome removes the vein and arteries.

In some embodiments, the umbilical cord is flattened and spread on a substrate (e.g., a frame backing paper). In some embodiments, the substrate comprises water-resistant coating or material. In some embodiments, the material is polyester. In some embodiments, the umbilical cord is orientated such that the inside face of the umbilical cord (e.g., the face comprising the Wharton's Jelly) faces up while the outside face (i.e., the face comprising amniotic membrane) contacts the substrate. In some embodiments, the umbilical cord is oriented using forceps.

In some embodiments, if the umbilical cord does not lay flat against the substrate, additional cuts are made in the Wharton's Jelly. In some embodiments, part or all of the Wharton's Jelly is removed from the umbilical cord. In some embodiments, the desired thickness of the tissue graft determines how much of the Wharton's Jelly is removed. In some embodiments, the Wharton's Jelly is peeled from the umbilical cord in layers (e.g., using a set of forceps, hemostats). In some embodiments, the Wharton's Jelly is cut away (e.g., shaved) from the umbilical cord in sections. In some embodiments, a rotoblator (i.e., a catheter attached to a drill with a diamond coated burr) is utilized to remove the Wharton's Jelly. In some embodiments, a liposuction machine is utilized to remove the Wharton's Jelly. In some embodiments, a liquid under high pressure is applied to remove the Wharton's Jelly. In some embodiments, a brush is utilized to remove the Wharton's Jelly (e.g., a mechanized brush rotating under high speed).

In some embodiments, the umbilical cord is cut and prepared to form umbilical cord sheet. In some embodiments, the umbilical cord is cut into multiple sections (e.g., using a scalpel). In some embodiments, a section of the umbilical cord is then cut longitudinally (e.g., using a scalpel or scissors). In some embodiments, the section of the umbilical cord is not cut into halves. In some embodiments, the section of the umbilical cord is cut into two halves. In some embodiments, the umbilical cord sheet is cut into strips or threads.

The umbilical cord can be cut while practicing aseptic technique under a laminar flow hood. In some embodiments, the umbilical cord is cut into desired shapes and sizes with a scalpel using a ruler as a guide. In some embodiments, the umbilical cord is cut to about 1.5 cm by about 0.3 cm using a scalpel and ruler with grids. In some embodiments, the umbilical cord is cut to about 0.5 cm×about 0.1 cm, 0.5 cm×about 0.25 cm, about 0.5 cm×about 0.5 cm, about 0.5 cm×about 0.75 cm, about 0.5 cm×about 1 cm, about 0.5 cm×about 2 cm, about 0.5 cm×about 3 cm, about 0.5 cm×about 4 cm, about 0.5 cm×about 5 cm, or greater. In some embodiments, the umbilical cord sheet is about 1 cm×about 0.1 cm, 1 cm×about 0.25 cm, about 1 cm×about 0.5 cm, about 1 cm×about 0.75 cm, about 1 cm×about 1 cm, about 1 cm×about 2 cm, about 1 cm×about 3 cm, about 1 cm×about 4 cm, about 1 cm×about 5 cm, or greater. In some embodiments, the umbilical cord sheet is about 1.5 cm×about 0.1 cm, 1.5 cm×about 0.25 cm, about 1.5 cm×about 0.5 cm, about 1.5 cm×about 0.75 cm, about 1.5 cm×about 1 cm, about 1.5 cm×about 2 cm, about 1.5 cm×about 3 cm, about 1.5 cm×about 4 cm, about 1.5 cm×about 5 cm, or greater using a scalpel and ruler with grids.

In some embodiments, the cut umbilical cord is stored in −20° C. In some embodiments, the umbilical cord is further subject to terminal sterilization by any suitable (e.g., medically acceptable) method including, but not limited to, gamma radiation, electron beam radiation, X-ray radiation, and UV radiation. In some embodiments, radiation is used with dry ice. In some, embodiments, radiation is used without dry ice. In some embodiments, the umbilical cord is exposed to radiation with dry ice for a first period of time and exposed to radiation without dry ice for a second period of time. In some embodiments, the umbilical cord is exposed to gamma radiation for a period of time sufficient to sterilize the umbilical cord. In some embodiments, the umbilical cord is exposed to electron beam (E-Beam) sterilization for a period of time sufficient to sterilize the umbilical cord. In some embodiments, the umbilical cord is exposed to electron beam (E-Beam) sterilization for a period of time sufficient to sterilize the umbilical cord. In some embodiments, the umbilical cord is further sterilized by gamma-irradiation at about 25 to about 43 kGy. In some embodiments, the umbilical cord is further sterilized by gamma-irradiation at about 10 to about 75 kGy. In some embodiments, the umbilical cord is further sterilized by gamma-irradiation at about 10, 15, 20, 25, 30, 35, 40, 45, 50, 55, 60, 65, 70, or more than 70 kGy. In some embodiments, the umbilical cord is exposed to electron beam (E-Beam) sterilization for a period of time sufficient to sterilize the umbilical cord. In some embodiments, the umbilical cord is further sterilized by E-Beam radiation at about 25 to about 43 kGy. In some embodiments, the umbilical cord is further sterilized by E-Beam radiation at about 10 to about 75 kGy. In some embodiments, the umbilical cord is further sterilized by E-Beam radiation at about 10, 15, 20, 25, 30, 35, 40, 45, 50, 55, 60, 65, 70, or more than 70 kGy. In some embodiments, the cut umbilical cord tissue is optionally washed again with buffer to further remove excess blood and tissue.

In some embodiments, the umbilical cord sheet comprises cells that are all, substantially all, or mostly dead. In some embodiments, the umbilical cord sheet is devoid of cells with metabolic activity. In some embodiments, the umbilical cord sheet comprises umbilical cord amniotic membrane and/or Wharton's jelly. In some embodiments, the umbilical cord sheet is partially or wholly free of Wharton's jelly. In some embodiments, the umbilical cord sheet is partially or wholly devoid of a vein or an artery. In some embodiments, the umbilical cord sheet is hydrated. In some embodiments, the umbilical cord sheet is obtained from frozen or previously-frozen umbilical cord.

In some embodiments, the umbilical cord sheet is substantially flattened. In some embodiments, the umbilical cord sheet is semi-circular, circular, rectangular, or tubular. In some embodiments, the umbilical sheet is a thread. In some embodiments, the umbilical cord sheet is a strip. In some embodiments, the umbilical sheet is configured to remain situated in the inferior fornix. In some embodiments, the umbilical sheet is configured to remain situated in the superior fornix. In some embodiments, the umbilical sheet is configured to remain situated in the inferior fornix and superior fornix. In some embodiments, the umbilical sheet is configured to dissolve over time. In some embodiments, the sheet dissolves over a period of about 2 days, about 3 days, about 4 days, about 5 days, about 6 days, about 7 days, about 10 days, about 2 weeks, about 3 weeks, about 1 month, or about 2 months.

In some embodiments, the umbilical cord sheet is about 0.5 cm×about 0.1 cm, 0.5 cm×about 0.25 cm, about 0.5 cm×about 0.5 cm, about 0.5 cm×about 0.75 cm, about 0.5 cm×about 1 cm, about 0.5 cm×about 2 cm, about 0.5 cm×about 3 cm, about 0.5 cm×about 4 cm, about 0.5 cm×about 5 cm, or greater. In some embodiments, the umbilical cord sheet is about 1 cm×about 0.1 cm, 1 cm×about 0.25 cm, about 1 cm×about 0.5 cm, about 1 cm×about 0.75 cm, about 1 cm×about 1 cm, about 1 cm×about 2 cm, about 1 cm×about 3 cm, about 1 cm×about 4 cm, about 1 cm×about 5 cm, or greater. In some embodiments, the umbilical cord sheet is about 1.5 cm×about 0.1 cm, 1.5 cm×about 0.25 cm, about 1.5 cm×about 0.5 cm, about 1.5 cm×about 0.75 cm, about 1.5 cm×about 1 cm, about 1.5 cm×about 2 cm, about 1.5 cm×about 3 cm, about 1.5 cm×about 4 cm, about 1.5 cm×about 5 cm, or greater. In some embodiments, the umbilical cord sheet is about 2 cm×about 0.1 cm, 2 cm×about 0.25 cm, about 2 cm×about 0.5 cm, about 2 cm×about 0.75 cm, about 2 cm×about 1 cm, about 2 cm×about 2 cm, about 2 cm×about 3 cm, about 2 cm×about 4 cm, about 2 cm×about 5 cm, or greater. In some embodiments, the umbilical cord sheet is about 2.5 cm×about 0.1 cm, 2.5 cm×about 0.25 cm, about 2.5 cm×about 0.5 cm, about 2.5 cm×about 0.75 cm, about 2.5 cm×about 1 cm, about 2.5 cm×about 2 cm, about 2.5 cm×about 3 cm, about 2.5 cm×about 4 cm, about 2.5 cm×about 5 cm, or greater. In some embodiments, the umbilical cord sheet is about 3 cm×about 0.1 cm, 3 cm×about 0.25 cm, about 3 cm×about 0.5 cm, about 3 cm×about 0.75 cm, about 3 cm×about 1 cm, about 3 cm×about 2 cm, about 3 cm×about 3 cm, about 3 cm×about 4 cm, about 3 cm×about 5 cm, or greater. In some embodiments, the umbilical cord sheet is about 1.0-2.0 cm by about 0.1-0.5 cm. In some embodiments, the umbilical cord sheet is about 1.5 cm by about 0.3 cm.

In some embodiments, the umbilical cord sheet is cut to a prescribed size using a scalpel. In some embodiments, the scalpel is disposable. In some embodiments, the scalpel is reusable. In some embodiments, the scalpel is combined with a ruler to obtain a straight cut of the umbilical cord. In some embodiments, the umbilical cord sheet is placed over a backing paper (e.g., PES backing paper), thereby reducing the tendency for the umbilical cord to slide while being cut. In some embodiments, the umbilical cord sheet is cut with scissors. In some embodiments, the scissors are reusable. In some embodiments, the scissors are disposable.

### Preparation of a HC-HA/PTX3 Complex

In some embodiments, the controlled release system comprises isolated HC-HA/PTX3 complex and an excipient for controlled release that is placed in the inferior fornix, superior fornix, onto a corneal surface, the punctum, or onto a tissue surrounding the eye of a subject thereby providing a controlled release of HC-HA/PTX3 into the tear film of the subject. In some embodiments, the controlled release system comprises isolated HC-HA/PTX3 complex and an excipient for controlled release that is placed in the inferior fornix only of the eye of a subject thereby providing a controlled release of HC-HA/PTX3 into the tear film of the subject.

In some embodiments, the isolated HC-HA/PTX3 is native HC-HA/PTX3 purified from a fetal support tissue. In some embodiments, the isolated HC-HA/PTX3 is reconstituted in vitro from its components (termed reconstituted HC-HA/PTX2 or rcHC-HA/PTX3). In some embodiments, the isolated HC-HA/PTX3 complex comprises native HC-HA/PTX3 (nHC-HA/PTX3) and reconstituted HC-HA/PTX3 (rcHC-HA/PTX3).

### Methods of Producing Isolated nHC-HA/PTX3 Complexes

In some embodiments, isolated native HC-HA/PTX3 (nHC-HA/PTX3) complexes are used in the methods provided herein.

In some embodiments, the isolated nHC-HA/PTX3 complex is isolated from an amniotic tissue. In some embodiments, the isolated nHC-HA/PTX3 complex is isolated from an amniotic membrane or an umbilical cord. In some embodiments, the isolated nHC-HA/PTX3 complex is isolated from fresh, frozen or previously frozen placental amniotic membrane (PAM), fresh, frozen or previously frozen umbilical cord amniotic membrane (UCAM), fresh, frozen or previously frozen placenta, fresh, frozen or previously frozen umbilical cord, fresh, frozen or previously frozen chorion, fresh, frozen or previously frozen amnion-chorion, or any combinations thereof. Such tissues can be obtained from any mammal, such as, for example, but not limited to a human, non-human primate, cow or pig.

In some embodiments, the nHC-HA/PTX3 is purified by any suitable method. In some embodiments, the nHC-HA/PTX3 complex is purified by centrifugation (e.g., ultracentrifugation, gradient centrifugation), chromatography (e.g., ion exchange, affinity, size exclusion, and hydroxyapatite chromatography), tangential flow filtration (TFF), gel filtration, or differential solubility, ethanol precipitation or by any other available technique for the purification of proteins (See, e.g., Scopes, Protein Purification Principles and Practice 2nd Edition, Springer-Verlag, New York, 1987; Higgins, S. J. and Hames, B. D. (eds.), Protein Expression: A Practical Approach, Oxford Univ Press, 1999; and Deutscher, M. P., Simon, M. I., Abelson, J. N. (eds.), Guide to Protein Purification: Methods in Enzymology (Methods in Enzymology Series, Vol 182), Academic Press, 1997, all incorporated herein by reference).

In some embodiments, the nHC-HA/PTX3 is isolated from an extract. In some embodiments, the extract is prepared from an amniotic membrane extract. In some embodiments, the extract is prepared from an umbilical cord extract. In some embodiments, the umbilical cord extract comprises umbilical cord stroma and/or Wharton's jelly. In some embodiments, the nHC-HA/PTX3 complex is contained in an extract that is prepared by ultracentrifugation. In some embodiments, the nHC-HA/PTX3 complex is contained in an extract that is prepared by ultracentrifugation using a CsCl/4-6M guanidine HCl gradient. In some embodiments, the extract is prepared by at least 2 rounds of ultracentrifugation. In some embodiments, the extract is prepared by more than 2 rounds of ultracentrifugation (i.e. nHC-HA/PTX3 2^{nd}). In some embodiments, the extract is prepared by at least 4 rounds of ultracentrifugation (i.e. nHC-HA/PTX3 4^{th}). In some embodiments, the nHC-HA/PTX3 complex comprises a small leucine-rich proteoglycan. In some embodiments, the nHC-HA/PTX3 complex comprises HC1, HA, PTX3 and/or a small leucine-rich proteoglycan.

In some embodiments, ultracentrifugation is performed on an extract prepared by extraction in an isotonic solution. In some embodiments, the isotonic solution is PBS. For example, in some embodiments the tissue is homogenized in PBS to produce a homogenized sample. The homogenized sample is then separated into a soluble portion and insoluble portion by centrifugation. In some embodiments, ultracentrifugation is performed on the soluble portion of the PBS-extracted tissue. In such embodiments, the nHC-HA/PTX3 purified by ultracentrifugation of the PBS-extracted tissue called an nHC-HA/PTX3 soluble complex. In some embodiments, the nHC-HA soluble complex comprises a small leucine-rich proteoglycan. In some embodiments, the nHC-HA/PTX3 soluble complex comprises HC1, HA, PTX3 and/or a small leucine-rich proteoglycan.

In some embodiments, ultracentrifugation is performed on an extract prepared by direct guanidine HCl extraction (e.g. 4-6 M GnHCl) of the amniotic membrane and/or umbilical cord tissue. In some embodiments, the GnHCl extract tissues is then centrifuged to produce GnHCl soluble and GnHCl insoluble portions. In some embodiments, ultracentrifugation is performed on the GnHCl soluble portion. In such embodiments, the nHC-HA/PTX3 purified by ultracentrifugation of the guanidine HCl-extracted tissue is called an nHC-HA/PTX3 insoluble complex. In some embodiments, the nHC-HA insoluble complex comprises a small leucine-rich proteoglycan. In some embodiments, the nHC-HA/PTX3 insoluble complex comprises HC1, HA, PTX3 and/or a small leucine-rich proteoglycan.

In some embodiments, ultracentrifugation is performed on an extract prepared by further guanidine HCl extraction of the insoluble portion of the PBS-extracted tissue. For example, in some embodiments the tissue is homogenized in PBS to produce a homogenized sample. The homogenized sample is then separated into a soluble portion and insoluble portion by centrifugation. The insoluble portion is then further extracted in guanidine HCl (e.g. 4-6 M GnHCl) and centrifuged to produce a guanidine HCl soluble and insoluble portions. In some embodiments, ultracentrifugation is performed on the guanidine HCl soluble portion. In such embodiments, the nHC-HA/PTX3 purified by ultracentrifugation of the guanidine HCl-extracted tissue is called an nHC-HA/PTX3 insoluble complex. In some embodiments, the nHC-HA insoluble complex comprises a small leucine-rich proteoglycan. In some embodiments, the nHC-HA/PTX3 insoluble complex comprises HC1, HA, PTX3 and/or a small leucine-rich proteoglycan.

In some embodiments, the method of purifying the isolated nHC-HA/PTX3 extract comprises: (a) dissolving the isolated extract (e.g. prepared by the soluble or insoluble method described herein) in CsCl/4-6M guanidine HCl at the initial density of 1.35 g/ml, to generate a CsCl mixture, (b) centrifuging the CsCl mixture at 125,000×g for 48 h at 15° C. to generate a first purified extract and pooling/adjusting HA-containing fractions to the initial density of 1.40 g/ml for the second ultracentrifugation at 125,000×g for 48 h at 15° C., (c) pooling the purified fractions and dialyzing against distilled water to remove CsCl and guanidine HCl, to generate a dialysate. In some embodiments, the method of purifying the isolated extract further comprises (d) mixing the dialysate with 3 volumes of 95% (v/v) ethanol containing 1.3% (w/v) potassium acetate at 0° C. for 1 h, to generate a first dialysate/ethanol mixture, (e) centrifuging the first dialysate/ethanol mixture at 15,000×g, to generate a second purified extract, and (f) extracting the second purified extract. In some embodiments, the method of purifying the isolated extract further comprises: (g) washing the second purified extract with ethanol (e.g., 70% ethanol), to generate a second purified extract/ethanol mixture; (h) centrifuging the second purified extract/ethanol mixture, to generate a third purified extract; and (i) extracting the third purified extract. In some embodiments, the method of purifying the isolated extract further comprises: (j) washing the third purified extract with ethanol (e.g., 70% ethanol), to generate a third purified extract/ethanol mixture; (k) centrifuging the third purified extract/ethanol mixture, to generate a forth purified extract; and (l) extracting the forth purified extract. In some embodiments, the purified extract comprises an nHC-HA/PTX3 complex.

In some embodiments, the nHC-HA/PTX3 complex is purified by immunoaffinity chromatography. In some embodiments, anti HC1 antibodies, anti-HC2 antibodies, or both are generated and affixed to a stationary support. In some embodiments, the unpurified HC-HA complex (i.e., the mobile phase) is passed over the support. In certain instances, the HC-HA complex binds to the antibodies (e.g., via interaction of (a) an anti-HC1 antibody and HC1, (b) an anti-HC2 antibody and HC2, (c) an anti-PTX antibody and PTX3, (d) an anti-SLRP antibody and the SLRP, or (e) any combination thereof). In some embodiments the support is washed (e.g., with PBS) to remove any unbound or loosely bound molecules. In some embodiments, the support is then washed with a solution that enables elution of the nHC-HA/PTX3 complex from the support (e.g., 1% SDS, 6M guanidine-HCl, or 8M urea).

In some embodiments, the nHC-HA/PTX3 complex is purified by affinity chromatography. In some embodiments, HABP is generated and affixed to a stationary support. In some embodiments, the unpurified nHC-HA/PTX3 complex (i.e., the mobile phase) is passed over the support. In certain instances, the nHC-HA/PTX3 complex binds to the HABP. In some embodiments the support is washed (e.g., with PBS) to remove any unbound or loosely bound molecules. In some embodiments, the support is then washed with a solution that enables elution of the HC-HA complex from the support.

In some embodiments, the nHC-HA/PTX3 complex is purified by a combination of HABP affinity chromatography, and immunoaffinity chromatography using anti HC1 antibodies, anti-HC2 antibodies, anti-PTX3 antibodies, antibodies against a SLRP or a combination of SLRPs, or any combination of antibodies thereof.

In some embodiments, the nHC-HA/PTX3 complex is purified from the insoluble fraction as described herein using one or more antibodies. In some embodiments, the nHC-HA/PTX3 complex is purified from the insoluble fraction as described herein using anti-SLRP antibodies.

In some embodiments, the nHC-HA/PTX3 complex is purified from the soluble fraction as described herein. In some embodiments, the nHC-HA/PTX3 complex is purified from the soluble fraction as described herein using anti-PTX3 antibodies.

In some embodiments, the nHC-HA/PTX3 complex comprises a small leucine rich proteoglycan (SLRP). In some embodiments, the nHC-HA/PTX3 complex comprises a class I, class II or class III SLRP. In some embodiments, the small leucine-rich proteoglycan is selected from among class I SLRPs, such as decorin and biglycan. In some embodiments, the small leucine-rich proteoglycan is selected from among class II SLRPs, such as fibromodulin, lumican, PRELP (proline arginine rich end leucine-rich protein), keratocan, and osteoadherin. In some embodiments, the small leucine-rich proteoglycan is selected from among class III SLRPs, such as epipycan and osteoglycin. In some embodiments, the small leucine-rich proteoglycan is selected from among bikunin, decorin, biglycan, and osteoadherin. In some embodiments, the small leucine-rich protein comprises a glycosaminoglycan. In some embodiments, the small leucine-rich proteoglycan comprises keratan sulfate.

### Methods of Producing Reconstituted HC-HA/PTX3 Complexes

In some embodiments, rcHC-HA/PTX3 complexes are used in the methods provided herein. Such reconstituted HC-HA/PTX3 complexes can be with or without SLRPs.

In some embodiments, a method for generating reconstituted HC-HA/PTX3 complexes comprises (a) contacting high molecular weight hyaluronan (HMW HA) with IαI and TSG-6 to form an HC-HA complex pre-bound to TSG-6 and (b) contacting the HC-HA complex with pentraxin 3 (PTX3) under suitable conditions to form an rcHC-HA/PTX3 complex. Provided herein are rcHC-HA/PTX3 complexes produced by such method. In some embodiments, HC1 of IαI forms a covalent linkage with HA. In some embodiments, the steps (a) and (b) of the method are performed sequentially in order. In some embodiments, the method comprises contacting an HC-HA complex pre-bound to TSG-6 with PTX3.

In some embodiments, the IαI protein and TSG-6 protein are contacted to the HMW HA at a molar ratio of about 1:1, 2:1, 3:1, 4:1, 5:1, 6:1, 7:1, 8:1, 9:1, 10:1, 15:1, or 20:1 (IαI:TSG-6). In some embodiments the ratio of IαI:TSG-6 ranges from about 1:1 to about 20:1, such as about 1:1 to about 10:1, such as about 1:1 to 5 about:1, such as about 1:1 to about 3:1. In some embodiments, the ratio of IαI:TSG-6 is 3:1 or higher. In some embodiments, the ratio of IαI:TSG-6 is 3:1.

In certain instances, TSG-6 interacts with IαI and forms covalent complexes with HC1 and HC2 of IαI (e.g., HC1⋅TSG-6 and HC2⋅TSG-6). In certain instances, in the presence of HA, the HCs are transferred to HA to form rcHC-HA.

In some embodiments, the step of contacting high molecular weight hyaluronan (HMW HA) with IαI and TSG-6 occurs for at least 10 minutes, at least 30 minutes, at least 1 hour, at least 2 hours, at least 3 hours, at least 4 hours, at least 5 hours, at least 6 hours, at least 12 hours, or at least 24 hours or longer. In some embodiments, the step of contacting BMW HA with IαI and TSG-6 occurs for at least 2 hours or longer. In some embodiments, the step of contacting HMW HA with IαI and TSG-6 occurs for at least 2 hours. In some embodiments, the step of contacting HMW HA with IαI and TSG-6 occurs at 37° C. In some embodiments, the step of contacting immobilized HMW HA with IαI and TSG-6 occurs in 5 mM MgCl₂ in PBS.

In some embodiments, the step of contacting PTX3 to an HC-HA complex occurs for at least 10 minutes, at least 30 minutes, at least 1 hour, at least 2 hours, at least 3 hours, at least 4 hours, at least 5 hours, at least 6 hours, at least 12 hours, or at least 24 hours or longer. In some embodiments, the step of contacting PTX3 to an HC-HA complex occurs for at least 2 hours or longer. In some embodiments, the step of contacting PTX3 to an HC-HA complex occurs for at least 2 hours. In some embodiments, the step of contacting PTX3 to an HC-HA complex occurs at 37° C. In some embodiments, the step of contacting PTX3 to an HC-HA complex occurs in 5 mM MgCl₂ in PBS.

In some embodiments, the method comprises contacting high molecular weight hyaluronan (HMW HA) with a pentraxin 3 (PTX3) protein, inter-α-inhibitor (IαI) protein comprising heavy chain 1 (HC1) and heavy chain 2 (HC2), and Tumor necrosis factor α-stimulated gene 6 (TSG-6) simultaneously under suitable conditions to form a HC-HA/PTX3 complex. In some embodiments, the contacting the HMW HA with PTX3, IαI and TSG-6 occurs for at least 10 minutes, at least 30 minutes, at least 1 hour, at least 2 hours, at least 3 hours, at least 4 hours, at least 5 hours, at least 6 hours, at least 12 hours, or at least 24 hours or longer. In some embodiments the step of contacting the HMW HA, PTX3, IαI, and TSG-6 occurs at 37° C. In some embodiments the step of contacting the HMW HA, PTX3, IαI, and TSG-6 occurs in 5 mM MgCl₂ in PBS.

In some embodiments, the method comprises contacting high molecular weight hyaluronan (HMW HA) with a pentraxin 3 (PTX3) protein, inter-α-inhibitor (IαI) protein comprising heavy chain 1 (HC1) and heavy chain 2 (HC2), and Tumor necrosis factor α-stimulated gene 6 (TSG-6) sequentially, in any order, under suitable conditions to form a HC-HA/PTX3 complex. In some embodiments, the contacting the HMW HA with PTX3, IαI and TSG-6 occurs for at least 10 minutes, at least 30 minutes, at least 1 hour, at least 2 hours, at least 3 hours, at least 4 hours, at least 5 hours, at least 6 hours, at least 12 hours, or at least 24 hours or longer. In some embodiments the step of contacting the HMW HA, PTX3, IαI, and TSG-6 occurs at 37° C. In some embodiments the step of contacting the HMW HA, PTX3, IαI, and TSG-6 occurs in 5 mM MgCl₂ in PBS.

In some embodiments, the methods for production of an rcHC-HA/PTX3 complex further comprises addition of one or more small leucine rich proteoglycans (SLRPs). In some embodiments, a method for generating reconstituted HC-HA/PTX3 complexes comprises (a) contacting high molecular weight hyaluronan (HMW HA) with IαI and TSG-6 to HA to form an HC-HA complex pre-bound to TSG-6, (b) contacting the HC-HA complex with pentraxin 3 (PTX3) and (c) contacting the HC-HA complex with one or more SLRPS under suitable conditions to form an rcHC-HA/PTX3 complex. Provided herein are rcHC-HA/PTX3 complexes produced by such method. In some embodiments, HCl of IαI forms a covalent linkage with HA. In some embodiments, the method comprises contacting an HC-HA complex pre-bound to TSG-6 with PTX3. In some embodiments, the steps (a), (b), and (c) of the method are performed sequentially in order. In some embodiments, the steps (a), (b), and (c) of the method are performed simultaneously. In some embodiments, the step (a) of the method is performed and then steps (b) and (c) of the method are performed sequentially in order. In some embodiments, the step (a) of the method is performed and then steps (b) and (c) of the method are performed simultaneously.

In some embodiments, the SLRP is selected from among a class I, class II or class II SLRP. In some embodiments, the SLRP is selected from among class I SLRPs, such as decorin and biglycan. In some embodiments, the small leucine-rich proteoglycan is selected from among class II SLRPs, such as fibromodulin, lumican, PRELP (proline arginine rich end leucine-rich protein), keratocan, and osteoadherin. In some embodiments, the small leucine-rich proteoglycan is selected from among class III SLRPs, such as epipycan and osteoglycin. In some embodiments, the small leucine-rich proteoglycan is selected from among bikunin, decorin, biglycan, and osteoadherin. In some embodiments, the small leucine-rich protein comprises a glycosaminoglycan. In some embodiments, the small leucine-rich proteoglycan comprises keratan sulfate.

### PTX3

In some embodiments, PTX3 for use in the methods is isolated from a cell or a plurality of cells (e.g., a tissue extract). Exemplary cells suitable for the expression of PTX3 include, but are not limited to, animal cells including, but not limited to, mammalian cells, primate cells, human cells, rodent cells, insect cells, bacteria, and yeast, and plant cells, including, but not limited to, algae, angiosperms, gymnosperms, pteridophytes and bryophytes. In some embodiments, PTX3 for use in the methods is isolated from a human cell. In some embodiments, PTX3 for use in the methods is isolated from a cell that is stimulated with one or more proinflammatory cytokines to upregulate PTX3 expression. In some embodiments, the proinflammatory cytokine is IL-1 or TNF-α.

In some embodiments, PTX3 for use in the methods is isolated from an amniotic membrane cell. In some embodiments, PTX3 for use in the methods is isolated from an amniotic membrane cell from an umbilical cord. In some embodiments, the amniotic membrane cell is stimulated with or more proinflammatory cytokines to upregulate PTX3 expression. In some embodiments, the proinflammatory cytokine is IL-1 or TNF-α.

In some embodiments, PTX3 for use in the methods is isolated from an umbilical cord cell. In some embodiments, the umbilical cord cell is stimulated with or more proinflammatory cytokines to upregulate PTX3 expression. In some embodiments, the proinflammatory cytokine is IL-1 or TNF-α.

In some embodiments, PTX3 for use in the methods is isolated from an amniotic epithelial cell. In some embodiments, PTX3 for use in the methods is isolated from an umbilical cord epithelial cell. In some embodiments, the amniotic epithelial cell or umbilical cord epithelial cell is stimulated with or more proinflammatory cytokines to upregulate PTX3 expression. In some embodiments, the proinflammatory cytokine is IL-1 or TNF-α.

In some embodiments, PTX3 for use in the methods is isolated from an amniotic stromal cell. In some embodiments, PTX3 for use in the methods is isolated from an umbilical cord stromal cell. In some embodiments, the amniotic stromal cell or umbilical cord stromal cell is stimulated with or more proinflammatory cytokines to upregulate PTX3 expression. In some embodiments, the proinflammatory cytokine is IL-1 or TNF-α.

In some embodiments, PTX3 for use in the methods is a native PTX3 protein isolated from a cell. In some embodiments, the cell is stimulated with or more proinflammatory cytokines to upregulate PTX3 expression. In some embodiments, the proinflammatory cytokine is IL-1 or TNF-α.

In some embodiments, PTX3 is prepared by recombinant technology. In some embodiments, PTX3 is expressed from a recombinant expression vector. In some embodiments, nucleic acid encoding PTX3 is operably linked to a constitutive promoter. In some embodiments, nucleic acid encoding PTX3 is operably linked to an inducible promoter. In some embodiments, PTX3 is expressed in a transgenic animal. In some embodiments, PTX3 is a recombinant protein. In some embodiments, PTX3 is a recombinant protein isolated from a cell. In some embodiments, PTX3 is a recombinant protein produced in a cell-free extract.

In some embodiments, PTX3 is purified from amniotic membrane, umbilical cord, umbilical cord amniotic membrane, chorionic membrane, amniotic fluid, or a combination thereof. In some embodiments, PTX3 is purified from amniotic membrane cells. In some embodiments, the amniotic membrane cell is an amniotic epithelial cell. In some embodiments, the amniotic membrane cell is an umbilical cord epithelial cell. In some embodiments, the amniotic membrane cell is an amniotic stromal cell. In some embodiments, the amniotic membrane cell is an umbilical cord stromal cell. In some embodiments, the amniotic membrane cell is stimulated with or more proinflammatory cytokines to upregulate PTX3 expression. In some embodiments, the proinflammatory cytokine is IL-1 or TNF-α.

In some embodiments, PTX3 is not isolated from a cell or a plurality of cells (e.g., a tissue extract).

In some embodiments, PTX3 comprises a fragment of PTX3 sufficient to facilitate the formation of rcHC-HA/PTX3 complex. Variants of PTX3 for use in the provided methods include variants with an amino acid modification that is an amino acid replacement (substitution), deletion or insertion. In some embodiments, such modification improves one or more properties of the PTX3 polypeptides such as improving the one or more therapeutic properties of the rcHC-HA/PTX3 complex (e.g., anti-inflammatory, anti-immune, anti-angiogenic, anti-scarring, anti-adhesion, regeneration or other therapeutic activities as described herein).

In some embodiments, PTX3 protein is obtained from a commercial source. An exemplary commercial source for PTX3 is, but is not limited to, PTX3 (Catalog No. 1826-TS; R&D Systems, Minneapolis, MN).

In some embodiments, the PTX3 protein used in the methods is a multimeric protein. In some embodiments, the PTX3 protein used in the methods is a homomultimer. In some embodiments, the homomultimer is a dimer, trimer, tetramer, hexamer, pentamer, or octamer. In some embodiments, the PTX3 homomultimer is a trimer, tetramer, or octamer. In particular embodiments, the PTX3 homomultimer is an octamer. In some embodiments, the multimerization domain is modified to improve multimerization of the PTX3 protein. In some embodiments, the multimerization domain is replaced with a heterogeneous multimerization domain (e.g., an Fc multimerization domain or leucine zipper) that when fused to PTX3 improves the multimerization of PTX3.

### TSG-6

In some embodiments, TSG-6 for use in the methods is isolated from a cell or a plurality of cells (e.g., a tissue extract). Exemplary cells suitable for the expression of TSG-6 include, but are not limited to, animal cells including, but not limited to, mammalian cells, primate cells, human cells, rodent cells, insect cells, bacteria, and yeast, and plant cells, including, but not limited to, algae, angiosperms, gymnosperms, pteridophytes and bryophytes. In some embodiments, TSG-6 for use in the methods is isolated from a human cell. In some embodiments, TSG-6 for use in the methods is isolated from a cell that is stimulated with one or more proinflammatory cytokines to upregulate TSG-6 expression. In some embodiments, the proinflammatory cytokine is IL-1 or TNF-α.

In some embodiments, TSG-6 for use in the methods is isolated from an amniotic membrane cell. In some embodiments, TSG-6 for use in the methods is isolated from an amniotic membrane cell from an umbilical cord. In some embodiments, TSG-6 for use in the methods is isolated from an amniotic membrane cell that is stimulated with one or more proinflammatory cytokines to upregulate TSG-6 expression. In some embodiments, the proinflammatory cytokine is IL-1 or TNF-α.

In some embodiments, TSG-6 for use in the methods is isolated from an umbilical cord cell. In some embodiments, TSG-6 for use in the methods is isolated from an umbilical cord cell that is stimulated with one or more proinflammatory cytokines to upregulate TSG-6 expression. In some embodiments, the proinflammatory cytokine is IL-1 or TNF-α.

In some embodiments, TSG-6 for use in the methods is isolated from an amniotic epithelial cell. In some embodiments, TSG-6 for use in the methods is isolated from an umbilical cord epithelial cell. In some embodiments, TSG-6 for use in the methods is isolated from an amniotic epithelial cell or an umbilical cord epithelial cell that is stimulated with one or more proinflammatory cytokines to upregulate TSG-6 expression. In some embodiments, the proinflammatory cytokine is IL-1 or TNF-α.

In some embodiments, TSG-6 for use in the methods is isolated from an amniotic stromal cell. In some embodiments TSG-6 for use in the methods is isolated from an umbilical cord stromal cell. In some embodiments, TSG-6 for use in the methods is isolated from an amniotic stromal cell or an umbilical cord stromal cell that is stimulated with one or more proinflammatory cytokines to upregulate TSG-6 expression. In some embodiments, the proinflammatory cytokine is IL-1 or TNF-α.

In some embodiments, TSG-6 for use in the methods is a native TSG-6 protein isolated from a cell. In some embodiments, the cell is stimulated with or more proinflammatory cytokines to upregulate TSG-6 expression. In some embodiments, the proinflammatory cytokine is IL-1 or TNF-α.

In some embodiments, TSG-6 is prepared by recombinant technology. In some embodiments, TSG-6 is expressed from a recombinant expression vector. In some embodiments, nucleic acid encoding TSG-6 is operably linked to a constitutive promoter. In some embodiments, nucleic acid encoding TSG-6 is operably linked to an inducible promoter. In some embodiments, TSG-6 is expressed in a transgenic animal. In some embodiments, TSG-6 is a recombinant protein. In some embodiments, TSG-6 is a recombinant protein isolated from a cell. In some embodiments, TSG-6 is a recombinant protein produced in a cell-free extract.

In some embodiments, TSG-6 is purified from amniotic membrane, amniotic membrane, chorionic membrane, amniotic fluid, or a combination thereof. In some embodiments, TSG-6 is purified from amniotic membrane cells. In some embodiments, the amniotic membrane cell is an amniotic epithelial cell. In some embodiments, the amniotic epithelial cell is an umbilical cord epithelial cell. In some embodiments, the amniotic membrane cell is an amniotic stromal cell. In some embodiments, the amniotic membrane cell is an umbilical cord stromal cell. In some embodiments, the amniotic membrane cell is stimulated with or more proinflammatory cytokines to upregulate TSG-6 expression. In some embodiments, the proinflammatory cytokine is IL-1 or TNF-α.

In some embodiments, TSG-6 is not isolated from a cell or a plurality of cells (e.g., a tissue extract).

In some embodiments, TSG-6 comprises a fragment of TSG-6 that is sufficient to facilitate or catalyze the transfer HC1 of IαI to HA. In some embodiments, TSG-6 comprises the link module of TSG-6.

In some embodiments, TSG-6 comprises an affinity tag. Exemplary affinity tags include but are not limited to a hemagglutinin tag, a poly-histidine tag, a myc tag, a FLAG tag, a glutathione-S-transferase (GST) tag. Such affinity tags are well known in the art for use in purification. In some embodiments, such an affinity tag incorporated into the TSG-6 polypeptide as a fusion protein or via a chemical linker. In some embodiments, TSG-6 comprises an affinity tag and the unbound TSG-6 is removed from the rcHC-HA/PTX3 complex by affinity purification.

In some embodiments TSG-6 protein is obtained from a commercial source. An exemplary commercial source for TSG-6 is, but is not limited to, TSG-6 (Catalog No. 2104-TS R&D Systems, Minneapolis, MN).

### IαI

In some embodiments, the IαI comprises an HC1 chain. In some embodiments, the IαI comprises an HC1 and an HC2 chain. In some embodiments, the IαI comprises an HC1, and HC2 chain and bikunin. In some embodiments, the IαI comprises an HC1, and HC2 chain and bikunin linked by a chondroitin sulfate chain.

In some embodiments, IαI is isolated from a biological sample. In some embodiments the biological sample is a biological sample from a mammal. In some embodiments, the mammal is a human. In some embodiments, the biological sample is a blood, serum, plasma, liver, amniotic membrane, chorionic membrane or amniotic fluid sample. In some embodiments, the biological sample is a blood, serum, or plasma sample. In some embodiments, the biological sample is a blood sample. In some embodiments, the biological sample is a serum sample. In some embodiments, the biological sample is a plasma sample. In some embodiments, the IαI is purified from human blood, plasma or serum. In some embodiments, IαI is isolated from human serum. In some embodiments, IαI is not isolated from serum. In some embodiments, IαI for use in the methods is produced in an amniotic membrane cell. In some embodiments, IαI for use in the methods is produced in an umbilical cord cell. In some embodiments, IαI for use in the methods is produced in an amniotic membrane cell from an umbilical cord. In some embodiments, IαI for use in the methods is produced in an amniotic epithelial cell. In some embodiments, IαI for use in the methods is produced in an umbilical cord epithelial cell. In some embodiments, IαI for use in the methods is produced in an amniotic stromal cell. In some embodiments, IαI for use in the methods is produced in an umbilical cord stromal cell. In some embodiments, IαI for use in the methods is produced in a hepatic cell. In some embodiments, IαI is prepared by recombinant technology.

In some embodiments, HC1 of IαI is isolated from a biological sample. In some embodiments the biological sample is a biological sample from a mammal. In some embodiments, the mammal is a human. In some embodiments, the biological sample is a blood, serum, plasma, liver, amniotic membrane, chorionic membrane or amniotic fluid sample. In some embodiments, the biological sample is a blood, serum, or plasma sample. In some embodiments, the biological sample is a blood sample. In some embodiments, the biological sample is a serum sample. In some embodiments, the biological sample is a plasma sample. In some embodiments, the HC1 of IαI is purified from human blood, plasma or serum. In some embodiments, IαI is isolated from human serum. In some embodiments, HC1 of IαI is not purified from serum. In some embodiments, HC1 of IαI is prepared by recombinant technology. In some embodiments, HC1 of IαI is purified from hepatic cells. In some embodiments, HC1 of IαI is purified from amniotic membrane cells. In some embodiments, HC1 of IαI is purified from amniotic epithelial cells or umbilical cord epithelial cells. In some embodiments, HC1 of IαI is purified from amniotic stromal cells or umbilical cord stromal cells.

In some embodiments, HC2 of IαI is isolated from a biological sample. In some embodiments the biological sample is a biological sample from a mammal. In some embodiments, the mammal is a human. In some embodiments, the biological sample is a blood, serum, plasma, liver, amniotic membrane, chorionic membrane or amniotic fluid sample. In some embodiments, the biological sample is a blood, serum, or plasma sample. In some embodiments, the biological sample is a blood sample. In some embodiments, the biological sample is a serum sample. In some embodiments, the biological sample is a plasma sample. In some embodiments, the HC2 of IαI is purified from human blood, plasma or serum. In some embodiments, HC2 of IαI is isolated from human serum. In some embodiments, HC2 of IαI is isolated from human serum. In some embodiments, HC2 of IαI is not isolated from blood serum. In some embodiments, HC2 of IαI is prepared by recombinant technology. In some embodiments, HC2 of IαI is purified from hepatic cells. In some embodiments, HC2 of IαI is purified from amniotic membrane cells. In some embodiments, HC2 of IαI is purified from amniotic epithelial cells or umbilical cord epithelial cells. In some embodiments, HC2 of IαI is purified from amniotic stromal cells or umbilical cord stromal cells.

### HA

In some embodiments, HA is purified from a cell, tissue or a fluid sample. In some embodiments, HA is obtained from a commercial supplier (e.g., Sigma Aldrich or Advanced Medical Optics, Irvine, CA (e.g., Healon)). In some embodiments, HA is obtained from a commercial supplier as a powder. In some embodiments, HA is expressed in a cell. Exemplary cells suitable for the expression of HA include, but are not limited to, animal cells including, but not limited to, mammalian cells, primate cells, human cells, rodent cells, insect cells, bacteria, and yeast, and plant cells, including, but not limited to, algae, angiosperms, gymnosperms, pteridophytes and bryophytes. In some embodiments, HA is expressed in a human cell. In some embodiments, HA is expressed in a transgenic animal. In some embodiments, HA is obtained from a cell that expresses a hyaluronan synthase (e.g., HAS1, HAS2, and HAS3). In some embodiments, the cell contains a recombinant expression vector that expresses an HA synthase. In certain instances, an HA synthase lengthens hyaluronan by repeatedly adding glucuronic acid and N-acetylglucosamine to the nascent polysaccharide as it is extruded through the cell membrane into the extracellular space.

HA for use in the methods is typically high molecular weight (HMW) HA. In some embodiments, the weight average molecular weight of HMW HA is greater than about 500 kilodaltons (kDa), such as, for example, between about 500 kDa and about 10,000 kDa, between about 800 kDa and about 8,500 kDa, between about 1100 kDa and about 5,000 kDa, or between about 1400 kDa and about 3,500 kDa. In some embodiments, the weight average molecular weight of HMW HA is about 3000 kDa.

### Preparation of a Controlled-Release Formulation Comprising an Isolated HC-HA/PTX3 Complex

In some embodiments, a controlled-release formulation comprises an isolated HC-HA/PTX3 complex mixed with an excipient for sustained release of HC-HA/PTX3. In some embodiments, the isolated HC-HA/PTX3 complex comprises native HC-HA/PTX3 (nHC-HA/PTX3) and/or reconstituted HC-HA/PTX3 (rcHC-HA/PTX3). In some embodiments, the excipient for sustained release of HC-HA/PTX3 is a polymer. In some embodiments, the excipient for sustained release of HC-HA/PTX3 is a biodegradable polymer. In some embodiments, the excipient for the controlled-release formulation comprises collagen, cellulose, chitosan, PEG, poly(N-isopropylacrylamide), poly(lactic) acid, poly(lactic-co-glycolic) acid, or a combination thereof. In some embodiments, the excipient for sustained release of HC-HA/PTX3 is configured to release the isolated HC-HA/PTX3 complex slowly and/or in a controlled manner, over time.

In some embodiments, an nHC-HA/PTX3 or rcHC-HA/PTX3 complex disclosed herein is formulated with one or more natural polymers. In some embodiments, an nHC-HA/PTX3 or rcHC-HA/PTX3 complex disclosed herein is formulated with a natural polymer that is fibronectin, collagen, laminin, keratin, fibrin, fibrinogen, hyaluronic acid, heparan sulfate, chondroitin sulfate, or combinations thereof. In some embodiments, an nHC-HA/PTX3 or rcHC-HA/PTX3 complex disclosed herein is formulated with a polymer gel formulated from a natural polymer. In some embodiments, an nHC-HA/PTX3 or rcHC-HA/PTX3 complex disclosed herein is formulated with a polymer gel formulated from a natural polymer, such as, but not limited to, fibronectin, collagen, laminin, keratin, fibrin, fibrinogen, hyaluronic acid, heparan sulfate, chondroitin sulfate, and combinations thereof. In some embodiments, an nHC-HA/PTX3 or rcHC-HA/PTX3 complex disclosed herein is formulated with a cross-linked polymer. In some embodiments, an nHC-HA/PTX3 or rcHC-HA/PTX3 complex disclosed herein is formulated with a non-cross-linked polymer. In some embodiments, an nHC-HA/PTX3 or rcHC-HA/PTX3 complex disclosed herein is formulated with a non-cross-linked polymer and a cross-linked polymer. In some embodiments, an nHC-HA/PTX3 or rcHC-HA/PTX3 complex disclosed herein is formulated with cross-linked hyaluronan gel. In some embodiments, an nHC-HA/PTX3 or rcHC-HA/PTX3 complex disclosed herein is formulated with an insoluble cross-linked HA hydrogel. In some embodiments, an nHC-HA/PTX3 or rcHC-HA/PTX3 complex disclosed herein is formulated with non-cross-linked hyaluronan gel. In some embodiments, an nHC-HA/PTX3 or rcHC-HA/PTX3 complex disclosed herein is formulated with a collagen matrix. In some embodiments, an nHC-HA/PTX3 or rcHC-HA/PTX3 complex disclosed herein is formulated with a fibrin matrix. In some embodiments, an nHC-HA/PTX3 or rcHC-HA/PTX3 complex disclosed herein is formulated with a fibrin/collagen matrix.

In some embodiments, an nHC-HA/PTX3 or rcHC-HA/PTX3 complex disclosed herein is formulated for administration to an eye or a tissue related thereto. Formulations suitable for administration to an eye include, but are not limited to, solutions, suspensions (e.g., an aqueous suspension), ointments, gels, creams, liposomes, niosomes, pharmacosomes, nanoparticles, or combinations thereof. In some embodiments, an nHC-HA/PTX3 or rcHC-HA/PTX3 complex disclosed herein for topical administration to an eye is administered spraying, washing, or combinations thereof.

In some embodiments, a formulation disclosed herein is in a dosage form. In some embodiments, a dosage form is liquid, solid, semi-solid or any combination thereof. In some embodiments, a semi-solid dosage form is for example a cream, gel, ointment, lotion, balm, suppository, topical forms or combinations thereof. In some embodiments, a solid dosage form is for example a tablet, capsule, granule, powder, sachet, reconstitutable powder, chewable, lozenge or any combination thereof.

In some embodiments, microencapsulated matrices (also known as microencapsulated matrices) of an nHC-HA/PTX3 or rcHC-HA/PTX3 complex disclosed herein are formed in in biodegradable polymers. In some embodiments, nHC-HA/PTX3 or rcHC-HA/PTX3 complex disclosed herein is entrapped in liposomes or microemulsions.

A formulation for administration to an eye has an ophthalmically acceptable tonicity. In certain instances, lacrimal fluid has an isotonicity value equivalent to that of a 0.9% sodium chloride solution. In some embodiments, an isotonicity value from about 0.6% to about1.8% sodium chloride equivalency is suitable for topical administration to an eye. In some embodiments, a formulation for administration to an eye disclosed herein has an osmolarity from about 200 to about 600 mOsm/L. In some embodiments, a formulation for administration to an eye disclosed herein is hypotonic and thus requires the addition of any suitable to attain the proper tonicity range. Ophthalmically acceptable substances that modulate tonicity include, but are not limited to, sodium chloride, potassium chloride, sodium thiosulfate, sodium bisulfite and ammonium sulfate.

A formulation for administration to an eye has an ophthalmically acceptable clarity. Examples of ophthalmically-acceptable clarifying agents include, but are not limited to, polysorbate 20, polysorbate 80, or combinations thereof.

In some embodiments, a formulation for administration to an eye comprises an ophthalmically acceptable viscosity enhancer. In some embodiments, a viscosity enhancer increases the time a formulation disclosed herein remains in an eye. In some embodiments, increasing the time a formulation disclosed herein remains in the eye allows for greater drug absorption and effect. Non-limiting examples of mucoadhesive polymers include carboxymethylcellulose, carbomer (acrylic acid polymer), polymethylmethacrylate), polyacrylamide, polycarbophil, acrylic acid/butyl acrylate copolymer, sodium alginate and dextran.

In some embodiments, the controlled-release formulation is configured to be placed in the inferior fornix, the superior fornix, the punctum, onto a corneal surface, or onto a tissue surrounding an eye of a subject. In some embodiments, the controlled-release formulation is configured to be placed in the inferior fornix only of an eye of a subject. In some embodiments, the controlled-release formulation is configured as a film and/or a coating. In some embodiments, the controlled-release formulation is flat or substantially flat. In some embodiments, the controlled-release formulation is configured to conform to the shape a corneal surface. In some embodiments, the controlled-release formulation is configured to remain coupled to the corneal surface. In some embodiments, the controlled-release formulation is configured to remain situated in the inferior fornix, the superior fornix, or both. In some embodiments, the controlled-release formulation is configured to dissolve over time. In some embodiments, the controlled-release formulation is configured to be transparent or translucent, thereby allowing the patient to see the controlled-release formulation is placed over the cornea.

In some embodiments, the controlled-release formulation is semi-circular, circular, rectangular, or tubular. In some embodiments, the controlled-release formulation is about 0.5 cm×about 0.1 cm, 0.5 cm×about 0.25 cm, about 0.5 cm×about 0.5 cm, about 0.5 cm×about 0.75 cm, about 0.5 cm×about 1 cm, about 0.5 cm×about 2 cm, about 0.5 cm×about 3 cm, about 0.5 cm×about 4 cm, about 0.5 cm×about 5 cm, or greater. In some embodiments, the controlled-release formulation is about 1 cm×about 0.1 cm, 1 cm×about 0.25 cm, about 1 cm×about 0.5 cm, about 1 cm×about 0.75 cm, about 1 cm×about 1 cm, about 1 cm×about 2 cm, about 1 cm×about 3 cm, about 1 cm×about 4 cm, about 1 cm×about 5 cm, or greater. In some embodiments, the controlled-release formulation is about 1.5 cm×about 0.1 cm, 1.5 cm×about 0.25 cm, about 1.5 cm×about 0.5 cm, about 1.5 cm×about 0.75 cm, about 1.5 cm×about 1 cm, about 1.5 cm×about 2 cm, about 1.5 cm×about 3 cm, about 1.5 cm×about 4 cm, about 1.5 cm×about 5 cm, or greater. In some embodiments, the controlled-release formulation is about 2 cm×about 0.1 cm, 2 cm×about 0.25 cm, about 2 cm×about 0.5 cm, about 2 cm×about 0.75 cm, about 2 cm×about 1 cm, about 2 cm×about 2 cm, about 2 cm×about 3 cm, about 2 cm×about 4 cm, about 2 cm×about 5 cm, or greater. In some embodiments, the controlled-release formulation is about 2.5 cm×about 0.1 cm, 2.5 cm×about 0.25 cm, about 2.5 cm×about 0.5 cm, about 2.5 cm×about 0.75 cm, about 2.5 cm×about 1 cm, about 2.5 cm×about 2 cm, about 2.5 cm×about 3 cm, about 2.5 cm×about 4 cm, about 2.5 cm×about 5 cm, or greater. In some embodiments, the controlled-release formulation is about 3 cm×about 0.1 cm, 3 cm×about 0.25 cm, about 3 cm×about 0.5 cm, about 3 cm×about 0.75 cm, about 3 cm×about 1 cm, about 3 cm×about 2 cm, about 3 cm×about 3 cm, about 3 cm×about 4 cm, about 3 cm×about 5 cm, or greater. In some embodiments, the controlled-release formulation is about 1.0-2.0 cm by about 0.1-0.5 cm. In some embodiments, the controlled-release formulation is about 1.5 cm by about 0.3 cm.

### Methods of Use

Disclosed herein, in some embodiments, are methods for treating an ocular surface disease, an ocular disorder, an ocular wound, or any combination thereof. In some embodiments, said method comprises placing an umbilical cord sheet in the inferior fornix, the superior fornix, the punctum, onto a corneal surface, or onto a tissue surrounding the eye of a subject having an ocular surface disease, disorder, or wound. In some embodiments, said method comprises placing an umbilical cord sheet in the inferior fornix only of the eye of a subject having an ocular surface disease, disorder, or wound. Further disclosed herein, in some embodiments, are methods for treating recurrent corneal erosion (RCE), corneal ulcers, herpes simplex keratitis (HSK), or OSD with recalcitrant punctate keratitis. As disclosed herein, the terms placing and applying are interchangeable. In some embodiments, said method comprises placing a controlled-release formulation, as disclosed herein, in an inferior fornix of an eye, the superior fornix, the punctum, onto a corneal surface, or onto a tissue surrounding the eye. In some embodiments, said method comprises placing a controlled-release formulation, as disclosed herein, in an inferior fornix only of an eye. In some embodiments, the method further comprises retracting a lower eyelid of the subject to expose the inferior fornix, the superior fornix, or both prior to placing the umbilical cord sheet and/or controlled-release formulation. In some embodiments, the method further comprises retracting an upper eyelid of the subject to expose the inferior fornix, the superior fornix, or both prior to placing the umbilical cord sheet and/or controlled-release formulation. In some embodiments, the method further comprises retracting an upper and lower eyelid of the subject to expose the inferior fornix, the superior fornix, or both prior to placing the umbilical cord sheet and/or controlled-release formulation. See FIG. 4. In some embodiments, the method comprises patching and/or taping an eye of a subject closed after applying the umbilical cord sheet and/or controlled-release formulation. In any method disclosed herein, an umbilical cord strip, an umbilical cord sheet, a controlled-release formulation comprising an excipient mixed with HC-HA/PTX3, or any combination thereof is placed in an inferior fornix, a superior fornix, the punctum, onto a corneal surface, or onto a tissue surrounding the eye to treat an ocular surface disease, disorder, or wound.

In some embodiments, the ocular surface disease or disorder is dry eye disease, recurrent corneal erosion (RCE), corneal ulcers, herpes simplex keratitis (HSK), superficial punctate keratitis (SPK), recalcitrant punctate keratitis, allergic conjunctivitis, red eye, pterygium, inflammatory disease, infectious diseases, or any combination thereof. In some embodiments, the ocular wound is a surgical wound. In some embodiments, the ocular wound is from Photorefractive keratectomy (PRK), laser-assisted in situ keratomileusis (LASIK), corneal cross-linking (CXL), corneal transplantation, cataract surgery, retinal surgeries, and/or a glaucoma drainage device or filtering bleb. In some embodiments, the ocular wound is from an injury, burn, and/or laceration.

In some embodiments, the ocular surface disease or disorder is superficial punctate keratitis (SPK), mild or moderate dry eye disease (DED), chronic ocular surface breakdown, epithelial healing, persistent keratitis, keratitis, severe dry eye disease (DED), Penetrating Keratoplasty (PKP), pemphigoid, limbal stem cell deficiency (LSCD), Stevens-Johnson syndrome (SJS), corneal abrasion, neurotrophic cornea, superficial keratectomy with epithelial basement membrane dystrophy (EBMD), corneal ulcer, recurrent corneal erosion, graft versus host disease (GVHD), filamentary keratitis, methicillin-resistant Staphylococcus aureus (MRSA), corneal persistent epithelial defects (PED), recurrent epithelial defect, or inflammatory interstitial keratitis. In some embodiments, the ocular surface disease or disorder is superficial punctate keratitis (SPK), mild or moderate dry eye disease (DED), chronic ocular surface breakdown, epithelial healing, persistent keratitis, or keratitis. In some embodiments, the ocular surface disease or disorder is severe dry eye disease (DED), Penetrating Keratoplasty (PKP), pemphigoid, limbal stem cell deficiency (LSCD), Stevens-Johnson syndrome (SJS), corneal abrasion, neurotrophic cornea, superficial keratectomy with epithelial basement membrane dystrophy (EBMD), corneal ulcer, recurrent corneal erosion, graft versus host disease (GVHD), filamentary keratitis, methicillin-resistant Staphylococcus aureus (MRSA), corneal persistent epithelial defects (PED), recurrent epithelial defect, or inflammatory interstitial keratitis.

In some embodiments, the umbilical cord sheet and/or controlled-release formulation is retained in the eye using any suitable method. In some embodiments, the umbilical cord sheet and/or controlled-release formulation is retained in the inferior fornix, the superior fornix, or both of the eye using any suitable method. In some embodiments, the umbilical cord sheet and/or controlled-release formulation is retained using suture, glue, an adhesive, or combinations thereof. In some embodiments, the adhesive comprises cyanoacrylate, fibrin, polyethyelene glycols, modified chondroitin sulfates, acrylic copolymers, biodendrimers, or combinations thereof.

In some embodiments, the umbilical cord sheet and/or controlled-release formulation promotes nerve regeneration in an eye of the subject, via the controlled-release of HC-HA/PTX3. In some embodiments, the umbilical cord sheet and/or controlled-release formulation is anti-inflammatory, anti-scarring, anti-angiogenic, anti-adhesion, reduces pain, and/or promotes wound healing.

In some embodiments, the method further comprises applying one or more additional therapeutic agents and/or fetal support tissue products to the inferior fornix, the superior fornix, or both corneal surface and/or surrounding tissue of the eye of the subject. In some embodiments, the additional therapeutic agent is a steroid, an anti-glaucoma agent, an antibacterial agent, an anti-fungal agent, an anti-viral agent, an anti-inflammatory agent, any ophthalmic drop, an agent used for dry eye treatment (e.g., cyclosporin), or any combination thereof. In some embodiments, the therapeutic agent is applied to the eye, e.g. inferior fornix, superior fornix, the punctum, corneal surface and/or surrounding tissue of the eye, prior to placing an umbilical cord sheet and/or controlled-release formulation to the tissue and/or surrounding tissue of the eye, e.g., inferior fornix and/or corneal surface. In some embodiments, the additional fetal support tissue product is obtained from a placental amniotic membrane, placenta, chorion, umbilical cord, umbilical cord amniotic membrane or any combination thereof. In some embodiments, the additional fetal support tissue product is a sheet, strip, thread, gel, homogenate, extract, reconstituted powder, or any combination thereof. In some embodiments, the umbilical cord sheet and/or controlled-release formulation placed in the inferior fornix, the superior fornix, the punctum, onto the corneal surface, or onto tissue surrounding the eye is configured to allow for longer retention of therapeutic biologics in the tear film.

In some embodiments, the method comprises utilizing an umbilical cord sheet and/or a controlled-release formulation as disclosed herein, including any size, shape, configuration as disclosed herein.

In some embodiments, the umbilical cord sheet comprises cells that are all, substantially all, or most of which are dead. In some embodiments, the umbilical cord strip is free of cells with metabolic activity. In some embodiments, the umbilical cord strip comprises umbilical cord amniotic membrane and/or Wharton's jelly. In some embodiments, the umbilical cord strip is partially or wholly devoid of Wharton's jelly. In some embodiments, the umbilical cord strip is partially or wholly devoid of a vein or an artery. In some embodiments, the umbilical cord strip is hydrated. In some embodiments, the umbilical cord strip is obtained from frozen or previously-frozen umbilical cord.

In some embodiments, the umbilical cord strip and/or controlled-release formulation is substantially flattened. In some embodiments, the umbilical cord strip and/or controlled-release formulation is of any shape. In some embodiments, the umbilical cord strip and/or controlled-release formulation is semi-circular, circular, rectangular, or tubular. In some embodiments, the umbilical strip is a thread. In some embodiments, the umbilical cord strip is a sheet.

In some embodiments, the umbilical cord strip and/or controlled-release formulation is about 0.5 cm×about 0.1 cm, 0.5 cm×about 0.25 cm, about 0.5 cm×about 0.5 cm, about 0.5 cm×about 0.75 cm, about 0.5 cm×about 1 cm, about 0.5 cm×about 2 cm, about 0.5 cm×about 3 cm, about 0.5 cm×about 4 cm, about 0.5 cm×about 5 cm, or greater. In some embodiments, the umbilical cord strip is about 1 cm×about 0.1 cm, 1 cm×about 0.25 cm, about 1 cm×about 0.5 cm, about 1 cm×about 0.75 cm, about 1 cm×about 1 cm, about 1 cm×about 2 cm, about 1 cm×about 3 cm, about 1 cm×about 4 cm, about 1 cm×about 5 cm, or greater. In some embodiments, the umbilical cord strip is about 1.5 cm×about 0.1 cm, 1.5 cm×about 0.25 cm, about 1.5 cm×about 0.5 cm, about 1.5 cm×about 0.75 cm, about 1.5 cm×about 1 cm, about 1.5 cm×about 2 cm, about 1.5 cm×about 3 cm, about 1.5 cm×about 4 cm, about 1.5 cm×about 5 cm, or greater. In some embodiments, the umbilical cord strip is about 2 cm×about 0.1 cm, 2 cm×about 0.25 cm, about 2 cm×about 0.5 cm, about 2 cm×about 0.75 cm, about 2 cm×about 1 cm, about 2 cm×about 2 cm, about 2 cm×about 3 cm, about 2 cm×about 4 cm, about 2 cm×about 5 cm, or greater. In some embodiments, the umbilical cord strip is about 2.5 cm×about 0.1 cm, 2.5 cm×about 0.25 cm, about 2.5 cm×about 0.5 cm, about 2.5 cm×about 0.75 cm, about 2.5 cm×about 1 cm, about 2.5 cm×about 2 cm, about 2.5 cm×about 3 cm, about 2.5 cm×about 4 cm, about 2.5 cm×about 5 cm, or greater. In some embodiments, the umbilical cord strip is about 3 cm×about 0.1 cm, 3 cm×about 0.25 cm, about 3 cm×about 0.5 cm, about 3 cm×about 0.75 cm, about 3 cm×about 1 cm, about 3 cm×about 2 cm, about 3 cm×about 3 cm, about 3 cm×about 4 cm, about 3 cm×about 5 cm, or greater. In some embodiments, the umbilical cord strip is about 1.0-2.0 cm by about 0.1-0.5 cm. In some embodiments, the umbilical cord strip is about 1.5 cm by about 0.3 cm.

Disclosed herein, in some embodiments, are methods for treating an ocular surface disease, an ocular disorder, an ocular wound, or any combination thereof by placing an umbilical cord sheet described herein in the inferior fornix, the superior fornix, the punctum, onto a corneal surface, or onto a tissue surrounding the eye of a subject having an ocular surface disease, disorder, or wound. In some embodiments, methods described herein using the umbilical cord sheet described herein results in improved benefits. In some embodiments, tolerance, clinical benefits, patient comfort, or combinations thereof are improved. In some embodiments, tolerance, clinical benefits, patient comfort, or combinations thereof are improved by at least or about 5%, 10%, 15%, 20%, 25%, 30%, 35%, 40%, 45%, 50%, 55%, 60%, 65%, 70%, 75%, 80%, 85%, 90%, 95%, or more than 95%. In some embodiments, clinical benefits include, but are not limited to, increased vision, decreased symptoms, decreased signs, and improved comfort. In some embodiments, tolerance, clinical benefits, patient comfort, or combinations thereof are improved for mild indications, moderate indications, or severe indications.

### Kit for Treating Ocular Surface Disease, Disorder, and/or Wound

In some embodiments, disclosed herein, is a kit for treatment of an ocular surface disease, ocular disorder, and/or ocular wound. In some embodiments, said kit comprises an umbilical cord sheet and a device for delivering the umbilical cord sheet to be placed in an inferior fornix, the superior fornix, the punctum, onto a corneal surface, or onto a tissue surrounding the eye of a subject. In some embodiments, said kit comprises an umbilical cord sheet and a device for delivering the umbilical cord sheet to be placed in an inferior fornix only of the eye of a subject. In some embodiments, said kit comprises a controlled-release formulation and a device for delivering the controlled-release formulation to be placed in an inferior fornix, a superior fornix, onto a corneal surface, or onto a tissue surrounding of an eye of a subject. In some embodiments, the kit comprises both an umbilical cord sheet and a controlled-release formulation, wherein one or more devices are used to deliver the umbilical cord sheet and controlled-release formulation to be placed in an inferior fornix, a superior fornix, the punctum, onto a corneal surface, or onto a tissue surrounding an eye. In some embodiments, the device, included with the kit, comprises at least one storage unit configured to contain the umbilical cord sheet and/or controlled-release formulation. In some embodiments, the umbilical cord sheet and/or controlled-release formulation is stored in a solution within a storage unit.

In some embodiments, the device further comprises at least one channel operatively coupled to at least one storage unit, wherein the channel is configured such that the umbilical cord sheet or controlled-release formulation pass through the channel from the at least one storage unit. In some embodiments, each channel further comprises an opening, such that the umbilical cord sheet/or controlled-release formulation pass through the channel and through the opening, and are subsequently placed in an inferior fornix, a superior fornix, the punctum, onto a corneal surface, or onto tissue surrounding an eye of a subject. In some embodiments, each channel further comprises an opening, such that the umbilical cord sheet/or controlled-release formulation pass through the channel and through the opening, and are subsequently placed in an inferior fornix only of an eye of a subject. In some embodiments, the umbilical cord sheet or controlled-release formulation pass through the opening with a solution. In some embodiments, the umbilical cord sheet or controlled-release formulation are substantially separated from a respective solution before passing through the opening (as described further below).

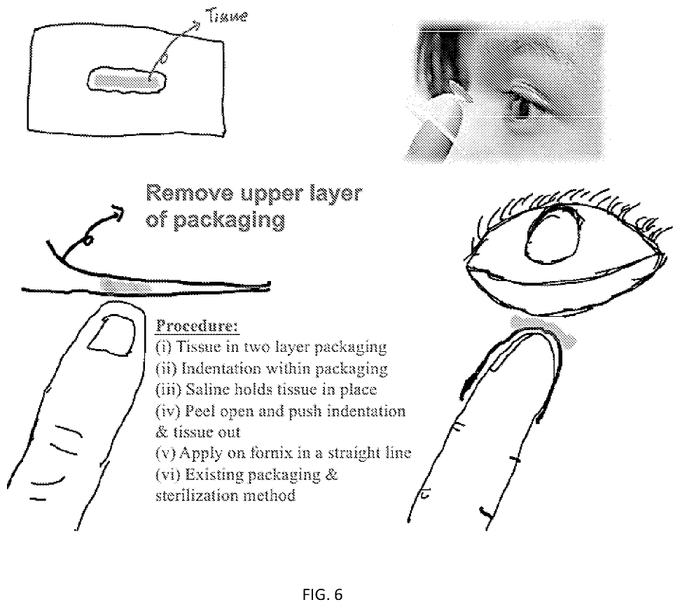

In some embodiments, the device or packaging for storing and applying the umbilical cord sheet or controlled-release formulation is as set forth in FIG. 6. As shown in FIG. 6, the packaging comprises two layers of packaging material (e.g., an upper and lower layer) with the umbilical cord sheet or controlled-release formulation disposed in the space between the two layers. In some embodiments, a solution such as saline holds the tissue in place. In some embodiments, the one layer of packaging comprises an indentation. In some embodiments, the upper layer is removed exposing the umbilical cord sheet, the indention in the lower layer of packaging is pushed from underneath the lower layer such that the packaging conforms to the fingertip, exposing the umbilical cord sheet on the fingertip. The umbilical cord sheet is then applied to the fornix using the fingertip.

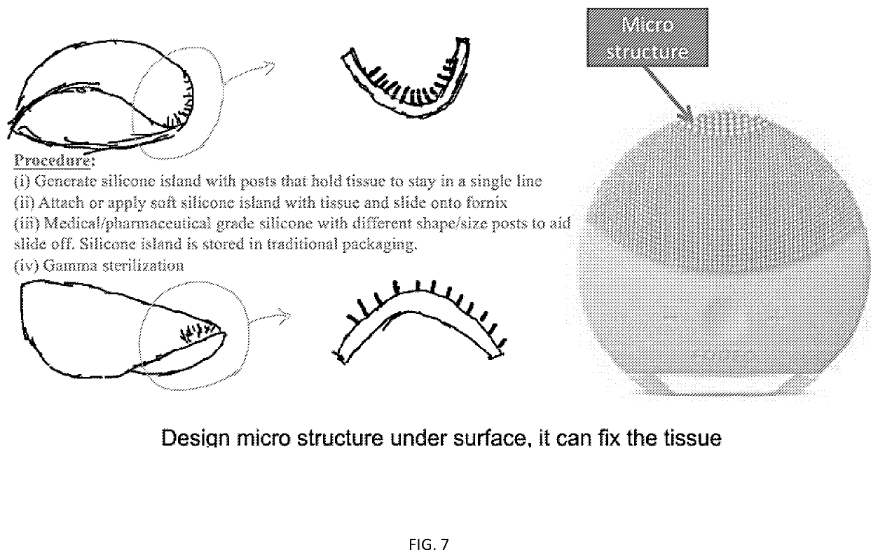

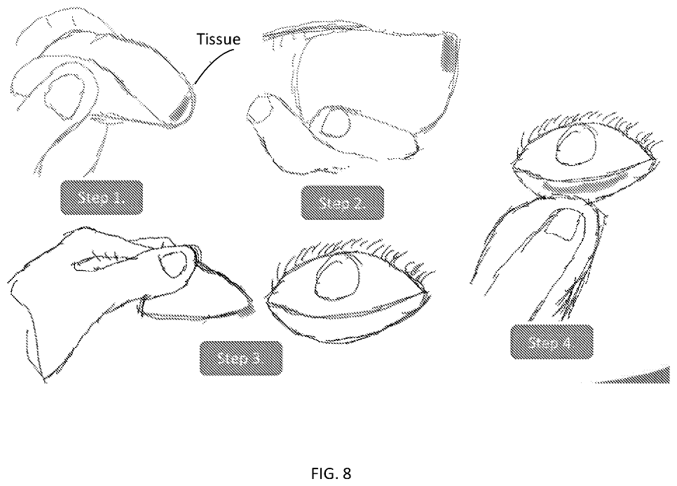

In some embodiments, the device or packaging for storing and applying the umbilical cord sheet or controlled-release formulation is as set forth in FIG. 7. As shown in FIG. 7, the packaging comprises a silicone island with micro structures or protrusions or posts that hold the umbilical cord sheet in place. The silicone island can be folded to expose the umbilical cord sheet which can be applied directly to the inferior fornix (FIG. 8).

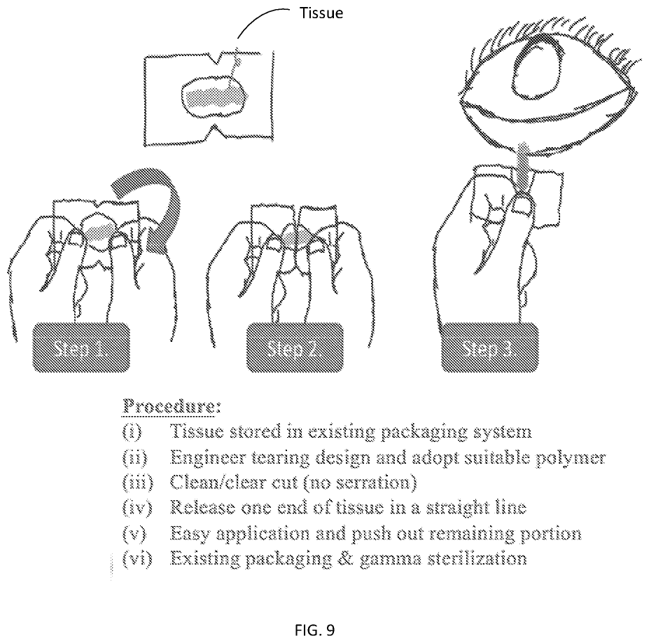

In some embodiments, the device or packaging for storing and applying the umbilical cord sheet or controlled-release formulation is as set forth in FIG. 9. As shown in FIG. 9, the packaging comprises the umbilical cord sheet in a shallow well of storage solution. The packaging comprises a tearing design or perforation so that the packaging may be opened exposing one end of the umbilical cord sheet which may be applied to the inferior fornix and the portion of the tissue remaining in the packaging may be pushed out.

In some embodiments, the packaging serves to store the umbilical cord sheet and to deliver the sheet directly by a physician into the inferior fornix, the superior fornix, or both without another tool. In some embodiments, the packaging is clear/transparent, non-biodegradable, durable, non-leachable, sterilizable by gamma-irradiation, biocompatible, and that meets USP 87 and/or ISO 10993 to package (with a sealing mechanism) and storage at room temperature or low refrigeration temperature of one or two umbilical cord strips, each with the dimension of 1.5×0.3 cm with approximately 200 μl of the saline. In some embodiments, the packaging material is designed to provide: (1) an opening to allow for packaging and sealing, (2) squeezable or manipulatable to allow the umbilical cord strip but not the excipient to come from a storage compartment through an outlet.

In some embodiments, the kit further comprises an additional therapeutic agent, wherein one or more devices are used to place the umbilical cord sheet, the controlled-release formulation, and/or the additional therapeutic agent in the inferior fornix, the superior fornix, the punctum, onto a corneal surface, or onto a tissue surrounding the eye. In some embodiments, the device is configured to place the therapeutic agent in the inferior fornix, the superior fornix, the punctum, onto a corneal surface, or onto a tissue surrounding the eye prior to applying the umbilical cord sheet and/or controlled-release formulation. In some embodiments, the therapeutic agent is a steroid, an anti-glaucoma agent, an antibacterial agent, an anti-fungal agent, an anti-viral agent, an anti-inflammatory agent, any ophthalmic drop, an agent used for dry eye treatment (e.g., cyclosporin), or any combination thereof.

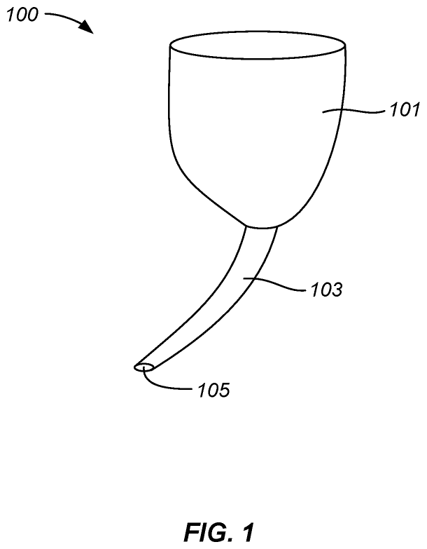

FIG. 1 depicts a non-limiting example of an embodiment of a device 100, as part of a kit, for delivering an umbilical cord sheet or a controlled-release formulation to an inferior fornix, a superior fornix, the punctum, corneal surface, or tissue surrounding an eye of a subject. In some embodiments, the device 100 comprises a storage unit 101 for storing the umbilical cord sheet or controlled-release formulation. In some embodiments, the storage unit 101 is operatively coupled to a channel 103, thereby enabling the umbilical cord sheet or controlled-release formulation to enter and pass through the channel 103 from the storage unit 101. In some embodiments, the channel 103 has an opening 105 configured such that the umbilical cord sheet or controlled-release formulation pass through said opening 105 and are delivered for placement into the inferior fornix, the superior fornix, the punctum, onto a corneal surface, or tissue surrounding an eye of a subject. In some embodiments, the umbilical cord sheet or controlled-release formulation slide out of the opening 105. In some embodiments, the umbilical cord sheet or controlled-release formulation flows out of the opening 105.

In some embodiments, the storage unit 101 is a pouch. In some embodiments, the storage unit 101 is transparent to allow visibility of the contents therein. In some embodiments, the storage unit 101 is free of holes, tears, creases or any other defect that compromises the storage unit 101 integrity. In some embodiments, the storage unit 101 comprises polycaprolactone, polyglycolic acid, poly(L-lactide), polylactic acid, ethylene propylene, olefinic thermoplastic elastomer, polyamide thermoplastic elastomer, silicones, polystyrene, polyurethane, chlorotrifluoroethylene, fluorinated ethylene propylene, perfluoroalkoxy, tetrafluoroethylene, polyvinyl fluoride, polyvinylidene difluoride, polysulfones, polyester, polybutylene terephthalate, and/or polyethylene terephthalate. In some embodiments, the storage unit 101 comprises material compatible with terminal Gamma-irradiation. In some embodiments, the storage unit 101 is sized to hold a prescribed amount of buffer solution and the umbilical cord sheet and/or controlled-release formulation. In some embodiments, the storage unit 101 is sized to hold between about 100 μL to about 1000 μL of buffer solution and an umbilical cord sheet cut to a dimension as disclosed herein. In some embodiments, the storage unit 101 is sized to hold between about 150 μL to about 500 μL of buffer solution and an umbilical cord sheet cut to a dimension as disclosed herein. In some embodiments, the storage unit 101 is sized to hold between about 200 μL to about 300 μL of buffer solution and an umbilical cord sheet cut to a dimension as disclosed herein.

In some embodiments, the storage unit 101 contains isotonic buffer and/or media to facilitate the movement of the umbilical cord sheet or controlled-release formulation, and/or to prevent drying of the umbilical cord sheet or controlled-release formulation. In some embodiments, the buffer is a saline solution. In some embodiments, the buffer and/or media is PBS, TRIS-buffered saline, HEPES-buffered saline, Ringer's solution, Hartmann's solution, EBSS, HBSS, Tyrode's Salt Solution, Gey's Balanced Salt Solution, DMEM, EMEM, or GMEM. In some embodiments, the media is a preservation medium.

In some embodiments, the storage unit 101 is oriented such that the umbilical cord sheet or controlled-release formulation pass through the channel and through the opening. In some embodiments, the storage unit 101 is positioned about an eye of a subject, such that the umbilical cord sheet or controlled-release formulation pass through the channel opening and into an inferior fornix, into the superior fornix, the punctum, onto a corneal surface, or onto a tissue surrounding the eye. In some embodiments, the storage unit 101 is moved laterally from side to side of an eye, such that the umbilical cord sheet or controlled-release formulation are correctly positioned within the inferior fornix, the superior fornix, the punctum, onto the corneal surface, or onto a tissue surrounding the eye.

In some embodiments, the device further comprises an application member operatively connected to the channel, wherein the application member is configured to move or push the umbilical cord sheet or controlled-release formulation through the channel opening, so as to deliver the umbilical cord sheet or controlled-release formulation to the inferior fornix, the superior fornix, the punctum, corneal surface, or tissue surrounding an eye of a subject. In some embodiments, the channel is configured to restrict the movement of the umbilical cord sheet or controlled-release formulation, so as to restrict the delivery of the umbilical cord sheet or controlled-release formulation, and the corresponding buffer and/or media to an eye. In some embodiments, the channel is configured to partially or wholly separate the buffer and/or media from the corresponding umbilical cord sheet or controlled-release formulation. In some embodiments, the channel is configured to separate the buffer and/or media from the umbilical cord sheet or controlled-release formulation prior to or during delivery of the umbilical cord sheet or controlled-release formulation to the inferior fornix, the superior fornix, the punctum, to a corneal surface, or to a tissue surrounding the eye. In some embodiments, the channel comprises a porous barrier within, e.g., a filter, configured such that the buffer and/or media pass through, while the umbilical cord sheet or controlled-release formulation are inhibited from passing through. In some embodiments, one or more pore sizes of the pores disposed on the porous barrier is selected to be smaller than the smallest anticipated size of the umbilical cord sheet or controlled-release formulation.

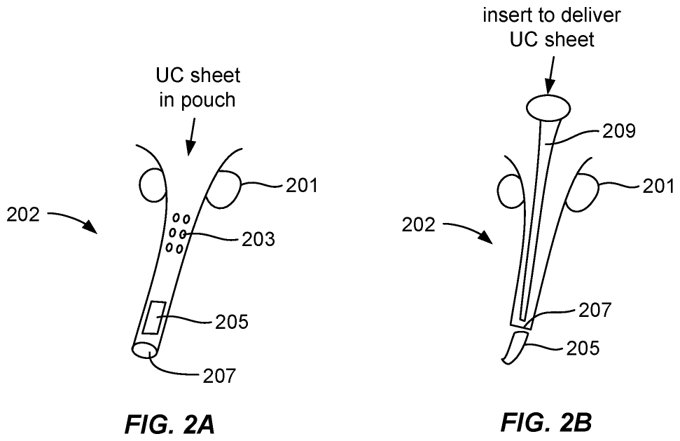

In some embodiments, the device comprises an application member that is configured to be inserted through the channel so as to deliver the umbilical cord sheet or controlled-release formulation for placement into the inferior fornix, the superior fornix, the punctum, onto a corneal surface, or tissue surrounding an eye of a subject. FIG. 2A depicts a non-limiting example of a channel 202 with finger rings 201 for easier handling of the device. The channel 202 comprises a barrier having one or more pores 203 configured to restrict and control the movement of the umbilical cord sheet or controlled-release formulation, and corresponding buffer and/or media, through the channel and through the opening 207. In some embodiments, at least one pore of the one or more pores 203 is configured and/or sized to inhibit or prevent the umbilical cord sheet or controlled-release formulation from passing through said pore. In some embodiments, the one or more pores inhibit the umbilical cord sheet or controlled-release formulation from passing through the channel. FIG. 2B depicts a non-limiting example of an application member 209 being inserted through the channel 202, and configured to push the umbilical cord sheet or controlled-release formulation 205 out of the opening 207, for delivery and placement into the inferior fornix, the superior fornix, the punctum, onto a corneal surface, or tissue surrounding an eye of a subject (not shown). In some embodiments, the application member is configured to push the umbilical cord sheet or controlled-release formulation through any pores within the channel, thereby enabling the umbilical cord sheet or controlled-release formulation to pass through the channel opening.

In some embodiments, the device is an injector comprising a vapor needle. In some embodiments, the injector comprises a housing for containing the umbilical cord sheet and/or controlled-release formulation. In some embodiments, the device is a syringe applicator, a graft syringe instrument, and/or a squeezable tube. In some embodiments, the graft implant syringe is a dental bone grafting instrument. In some embodiments, the device includes forceps for placement of the umbilical cord sheet and/or controlled-release formulation onto the inferior fornix, the superior fornix, the punctum, onto a corneal surface, or tissue surrounding an eye of a subject.

In some embodiments, the kit disclosed herein comprises an umbilical cord sheet and/or a controlled-release formulation as disclosed herein, including any size, shape, configuration, and so on. In some embodiments, the kit comprises a fetal support tissue powder or gel formulation.

In some embodiments, the umbilical cord tissue comprises cells that are all, substantially all, or mostly dead. In some embodiments, the umbilical cord tissue is devoid of cells with metabolic activity. In some embodiments, the umbilical cord tissue comprises umbilical cord amniotic membrane and/or Wharton's jelly. In some embodiments, the umbilical cord tissue is partially or wholly devoid of Wharton's jelly. In some embodiments, the umbilical cord tissue substantially devoid of a vein or an artery. In some embodiments, the umbilical cord tissue is hydrated. In some embodiments, the umbilical cord tissue is obtained from frozen or previously-frozen umbilical cord.

In some embodiments, the umbilical cord tissue and/or controlled-release formulation is substantially flattened. In some embodiments, the umbilical cord tissue and/or controlled-release formulation is of any shape. In some embodiments, the umbilical cord tissue and/or controlled-release formulation is semi-circular, circular, rectangular, or tubular. In some embodiments, the umbilical tissue is a strip. In some embodiments, the umbilical tissue is a thread. In some embodiments, the umbilical cord tissue is a sheet.

In some embodiments, the umbilical cord tissue and/or controlled-release formulation is about 0.5 cm×about 0.1 cm, 0.5 cm×about 0.25 cm, about 0.5 cm×about 0.5 cm, about 0.5 cm×about 0.75 cm, about 0.5 cm×about 1 cm, about 0.5 cm×about 2 cm, about 0.5 cm×about 3 cm, about 0.5 cm×about 4 cm, about 0.5 cm×about 5 cm, or greater. In some embodiments, the umbilical cord sheet is about 1 cm×about 0.1 cm, 1 cm×about 0.25 cm, about 1 cm×about 0.5 cm, about 1 cm×about 0.75 cm, about 1 cm×about 1 cm, about 1 cm×about 2 cm, about 1 cm×about 3 cm, about 1 cm×about 4 cm, about 1 cm×about 5 cm, or greater. In some embodiments, the umbilical cord sheet is about 1.5 cm×about 0.1 cm, 1.5 cm×about 0.25 cm, about 1.5 cm×about 0.5 cm, about 1.5 cm×about 0.75 cm, about 1.5 cm×about 1 cm, about 1.5 cm×about 2 cm, about 1.5 cm×about 3 cm, about 1.5 cm×about 4 cm, about 1.5 cm×about 5 cm, or greater. In some embodiments, the umbilical cord sheet is about 2 cm×about 0.1 cm, 2 cm×about 0.25 cm, about 2 cm×about 0.5 cm, about 2 cm×about 0.75 cm, about 2 cm×about 1 cm, about 2 cm×about 2 cm, about 2 cm×about 3 cm, about 2 cm×about 4 cm, about 2 cm×about 5 cm, or greater. In some embodiments, the umbilical cord sheet is about 2.5 cm×about 0.1 cm, 2.5 cm×about 0.25 cm, about 2.5 cm×about 0.5 cm, about 2.5 cm×about 0.75 cm, about 2.5 cm×about 1 cm, about 2.5 cm×about 2 cm, about 2.5 cm×about 3 cm, about 2.5 cm×about 4 cm, about 2.5 cm×about 5 cm, or greater. In some embodiments, the umbilical cord sheet is about 3 cm×about 0.1 cm, 3 cm×about 0.25 cm, about 3 cm×about 0.5 cm, about 3 cm×about 0.75 cm, about 3 cm×about 1 cm, about 3 cm×about 2 cm, about 3 cm×about 3 cm, about 3 cm×about 4 cm, about 3 cm×about 5 cm, or greater. In some embodiments, the umbilical cord sheet is about 1.0-2.0 cm by about 0.1-0.5 cm. In some embodiments, the umbilical cord sheet is about 1.5 cm by about 0.3 cm.

The kit comprising the umbilical cord can be used to treat ocular surface disease, disorder, or wound a subject. The ocular surface disease or disorder can be selected from the group consisting of dry eye disease, recurrent corneal erosion (RCE), corneal ulcers, herpes simplex keratitis (HSK), superficial punctate keratitis (SPK), recalcitrant punctate keratitis, allergic conjunctivitis, red eye, pterygium, and inflammatory disease. The wound can be a surgical wound. The wound can be a surgical wound. In some embodiments, the wound can be from photorefractive keratectomy (PRK), laser-assisted in situ keratomileusis (LASIK), corneal cross-linking (CXL), or a glaucoma drainage device and/or filtering bleb. In some embodiments, the wound is from an injury, burn, and/or laceration. In some embodiments, the ocular surface disease or disorder is superficial punctate keratitis (SPK), mild or moderate dry eye disease (DED), chronic ocular surface breakdown, epithelial healing, persistent keratitis, keratitis, severe dry eye disease (DED), Penetrating Keratoplasty (PKP), pemphigoid, limbal stem cell deficiency (LSCD), Stevens-Johnson syndrome (SJS), corneal abrasion, neurotrophic cornea, superficial keratectomy with epithelial basement membrane dystrophy (EBMD), corneal ulcer, recurrent corneal erosion, graft versus host disease (GVHD), filamentary keratitis, methicillin-resistant Staphylococcus aureus (MRSA), corneal persistent epithelial defects (PED), recurrent epithelial defect, or inflammatory interstitial keratitis. In some embodiments, the ocular surface disease or disorder is superficial punctate keratitis (SPK), mild or moderate dry eye disease (DED), chronic ocular surface breakdown, epithelial healing, persistent keratitis, or keratitis. In some embodiments, the ocular surface disease or disorder is severe dry eye disease (DED), Penetrating Keratoplasty (PKP), pemphigoid, limbal stem cell deficiency (LSCD), Stevens-Johnson syndrome (SJS), corneal abrasion, neurotrophic cornea, superficial keratectomy with epithelial basement membrane dystrophy (EBMD), corneal ulcer, recurrent corneal erosion, graft versus host disease (GVHD), filamentary keratitis, methicillin-resistant Staphylococcus aureus (MRSA), corneal persistent epithelial defects (PED), recurrent epithelial defect, or inflammatory interstitial keratitis.

In some embodiments, the umbilical cord sheet can promote nerve regeneration in the eye of the subject. In some embodiments, the umbilical cord sheet is anti-inflammatory, anti-scarring, anti-angiogenic, anti-adhesion, reduces pain, and/or promotes wound healing when contacted with an exogenous living cell. In some embodiments, the umbilical cord sheet has homologous use.

In some embodiments, the umbilical cord product kit further comprises one or more therapeutic agents and/or a fetal support tissue product. The therapeutic agent can be a steroid, an anti-glaucoma agent, an antibacterial agent, an anti-fungal agent, an anti-viral agent, and/or an anti-inflammatory agent. The fetal support tissue product can be taken from placental amniotic membrane, placenta, chorion, umbilical cord, umbilical cord amniotic membrane or any combination thereof. The fetal support tissue product can be a sheet, strip, or thread.

### Certain Terminology

As used herein and in the appended claims, the singular forms “a,” “and,” and “the” include plural referents unless the context clearly dictates otherwise.

Also, the use of “and” means “and/or” unless stated otherwise. Similarly, “comprise,” “comprises,” “comprising” “include,” “includes,” and “including” are interchangeable and not intended to be limiting.

As used herein, the term “about” refers to an amount that is near the stated amount, for example by 10%, 5%, or 1%, including increments therein.

The terms “subject” and “individual” are used interchangeably. As used herein, both terms mean any animal, preferably a mammal, including a human or non-human. The terms patient, subject, and individual are used interchangeably. None of the terms are to be interpreted as requiring the supervision of a medical professional (e.g., a doctor, nurse, physician's assistant, orderly, hospice worker).

As used herein, “placental amniotic membrane” (PAM) means amniotic membrane derived from the placenta. In some embodiments, the PAM is substantially isolated.

The terms “treat,” “treating” or “treatment,” as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

As used herein, “umbilical cord amniotic membrane” (UCAM) means amniotic membrane derived from the umbilical cord. UCAM is a translucent membrane. The UCAM has multiple layers an epithelial layer, a basement membrane; a compact layer; a fibroblast layer; and a spongy layer. It lacks blood vessels or a direct blood supply. In some embodiments, the UCAM is substantially isolated. In some embodiments, the UCAM comprises Wharton's Jelly. In some embodiments, the UCAM comprises blood vessels and/or arteries. In some embodiments, the UCAM comprises Wharton's Jelly and blood vessels and/or arteries.

### EXAMPLES

### Example 1: Preparation of Umbilical Cord Strip

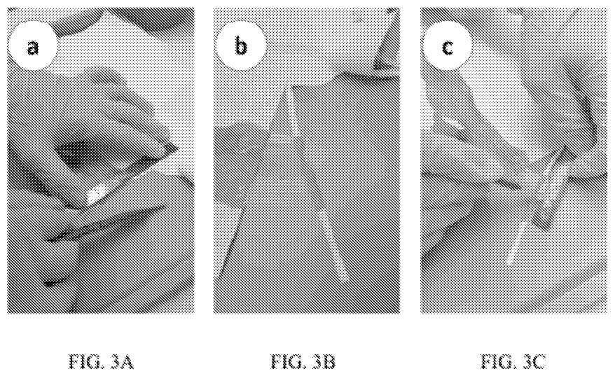

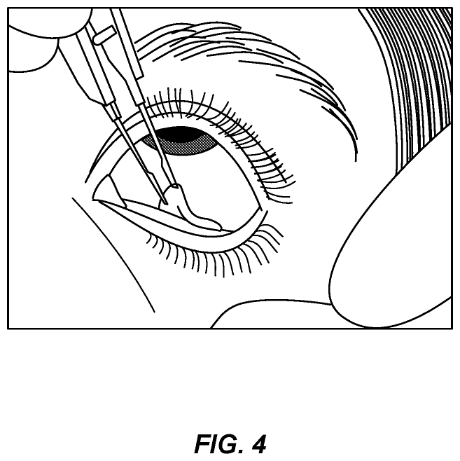

A frozen piece of umbilical cord tissue was obtained and thawed for about 5 min at ambient temperature. The umbilical cord tissue was transferred to a sterile tray under a laminar flow hood. The stroma side of the umbilical cord was placed face-down on a frame backing paper and flattened. A pair of forceps was used to manipulate the tissue. Practicing aseptic technique, the tissue was cut using a scalpel with a ruler as a measurement guide to form an umbilical cord sheet. See FIGS. 3A-3C. The umbilical cord sheet was sterilized by gamma-irradiation at 25-43 kGy with dry ice.

### Example 2: Treatment Post-PRK With Umbilical Cord Strip

An umbilical cord sheet of about 1.5 cm by 0.25 cm is prepared. The lower eyelid of a human subject who has undergone photorefractive keratectomy (PRK) is retracted and the umbilical cord strip is placed in the inferior fornix. The umbilical cord strip is kept in the subject's eye for 18 days and removed. The cornea is examined by a physician for healing progress.

### Example 3: Case Study of Treatment of Eye Post-PRK With Umbilical Cord Strip

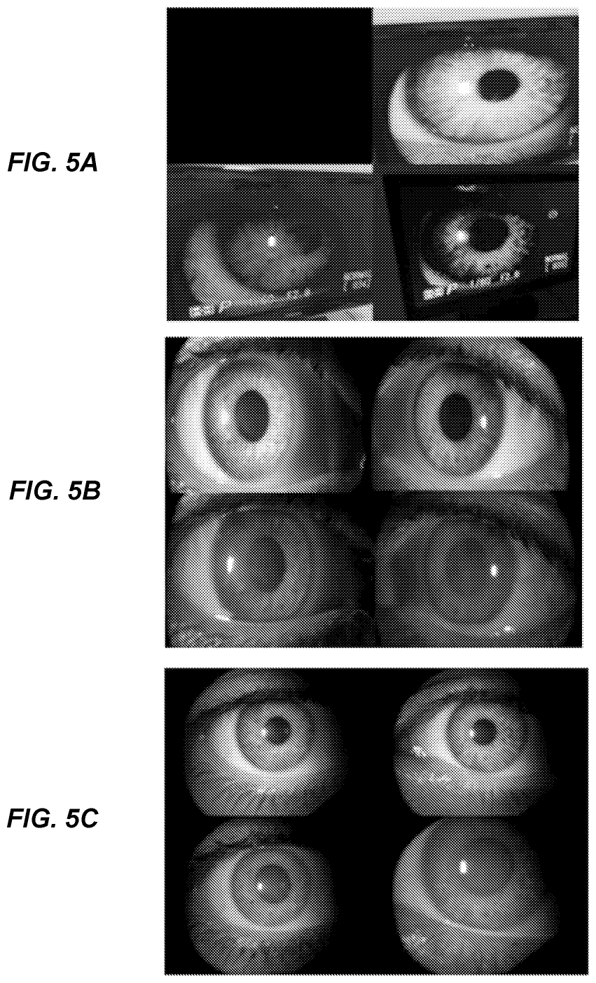

Patient underwent photorefractive keratectomy (PRK) on May 30. Following PRK, an umbilical cord sheet was placed in the inferior fornix of both eyes to help promote nerve regeneration, and prevent dry eye disease, which is common for patients who undergo PRK. Specifically, on Day 0, an umbilical cord sheet (NEOX CORD RT product) was cut using sterile technique into strips measuring approximately 1.5 cm by 0.25 cm. A topical anesthetic was first applied to patient's eyes. Subsequently, an umbilical cord strip was placed into the inferior fornix of each of Patient's eyes. Patient indicated no discomfort, pain, or foreign body sensation. On Day 1, the umbilical strip fell out of Patient's right eye. Subsequently, two umbilical cord strips were placed in the inferior fornix of Smith's right eye. On Day 2, Patient indicated no pain, discomfort, or foreign body sensation with the umbilical cord strips placed into the eyes, but indicated it was possible he subjectively detected a difference between 1 strip in his left eye, and 2 strips in his right eyes. On Day 15, Patient's eyes were evaluated, and it was noted the cornea appeared to be clear without any hazing, and that eyes had recovered from the PRK procedure. Slit-lamp photography and fluorescein staining was used to measure the epithelialization and corneal surface integrity. FIGS. 5A-5C depict the Days 0-2 of an umbilical cord sheet as positioned on the inferior fornix, viewed using the fluorescence.

### Example 4: Use of UC Strip in Dry Eye Disease (DED)

A UC strip is applied to the inferior fornix of one eye of a patient with refractory DED. At one month the eye is evaluated by a physician and compared to the to the untreated eye.

### Example 5: Use of UC Strip for Ocular Disease

UC strips were used for subjects with mild/moderate and severe indications including superficial punctate keratitis (SPK), mild or moderate dry eye disease (DED), chronic ocular surface breakdown, epithelial healing, persistent keratitis, keratitis, severe dry eye disease (DED), Penetrating Keratoplasty (PKP), pemphigoid, limbal stem cell deficiency (LSCD), Stevens-Johnson syndrome (SJS), corneal abrasion, neurotrophic cornea, superficial keratectomy with epithelial basement membrane dystrophy (EBMD), corneal ulcer, recurrent corneal erosion, graft versus host disease (GVHD), filamentary keratitis, methicillin-resistant Staphylococcus aureus (MRSA), corneal persistent epithelial defects (PED), recurrent epithelial defect, or inflammatory interstitial keratitis.

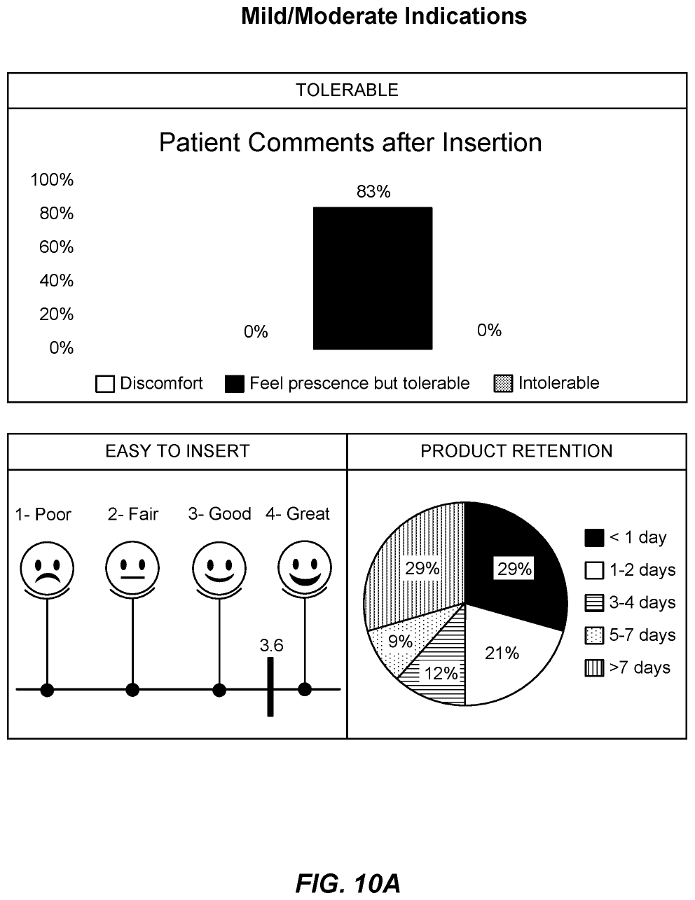

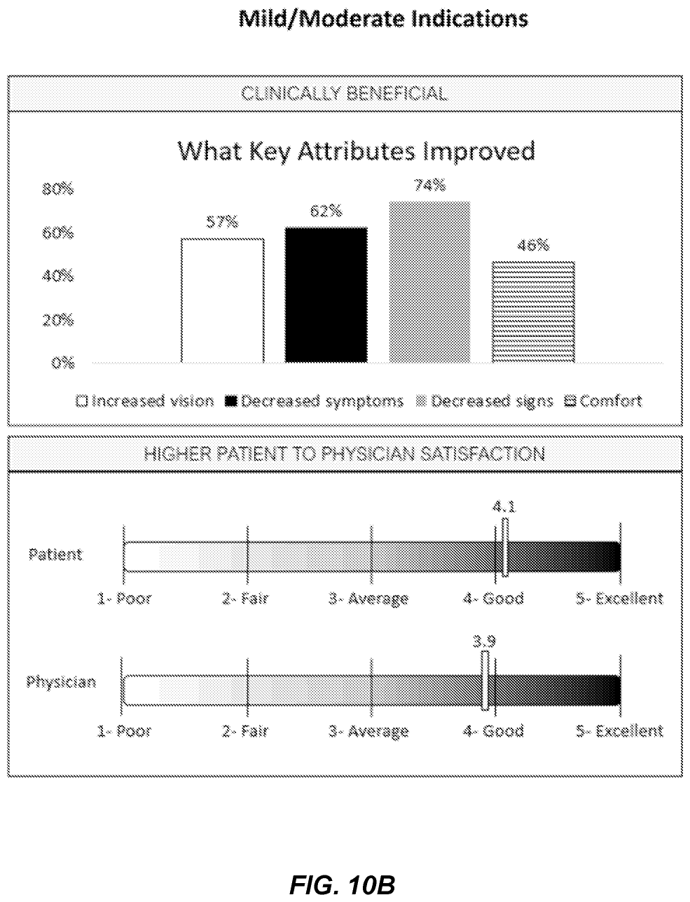

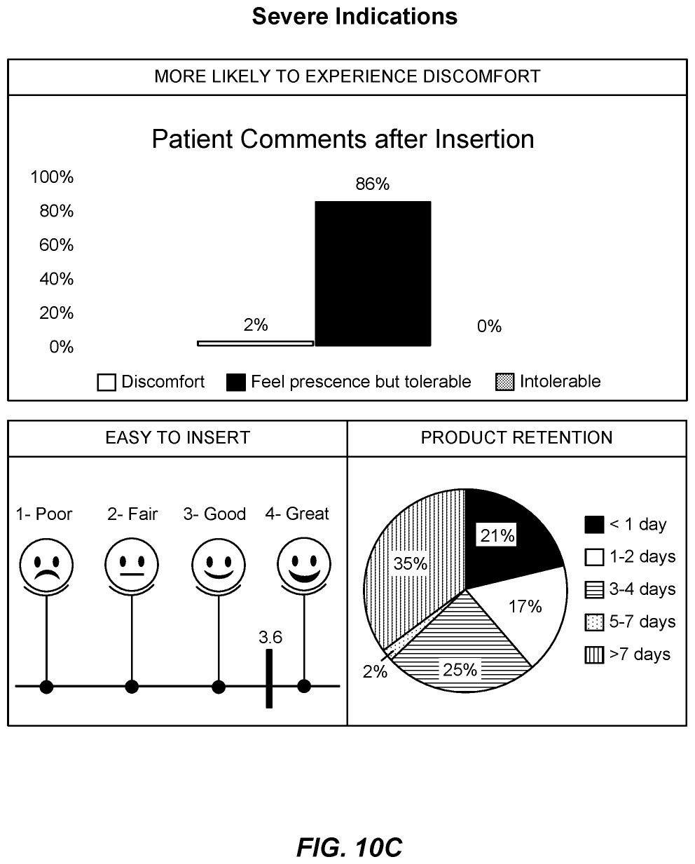

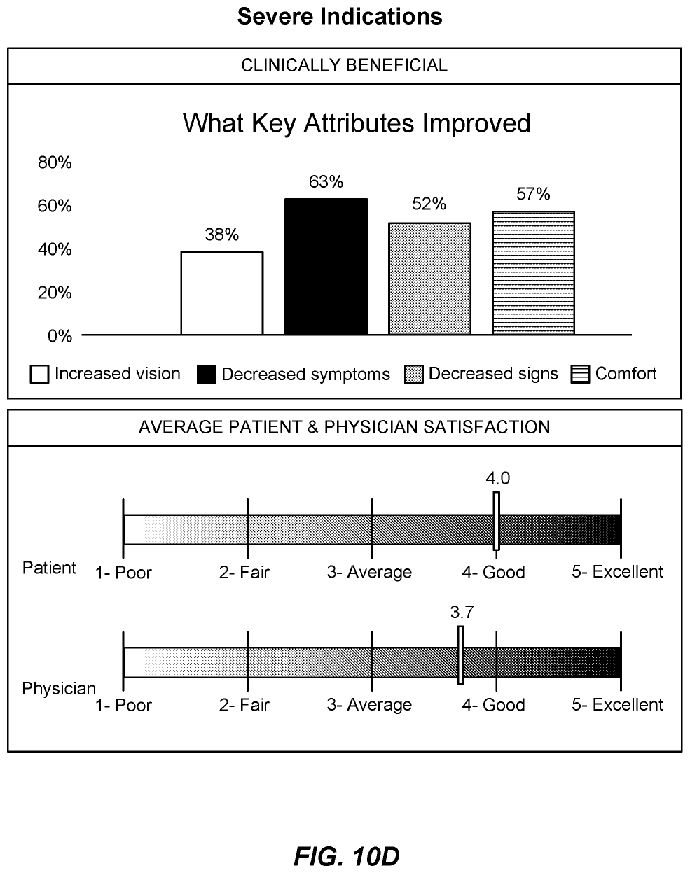

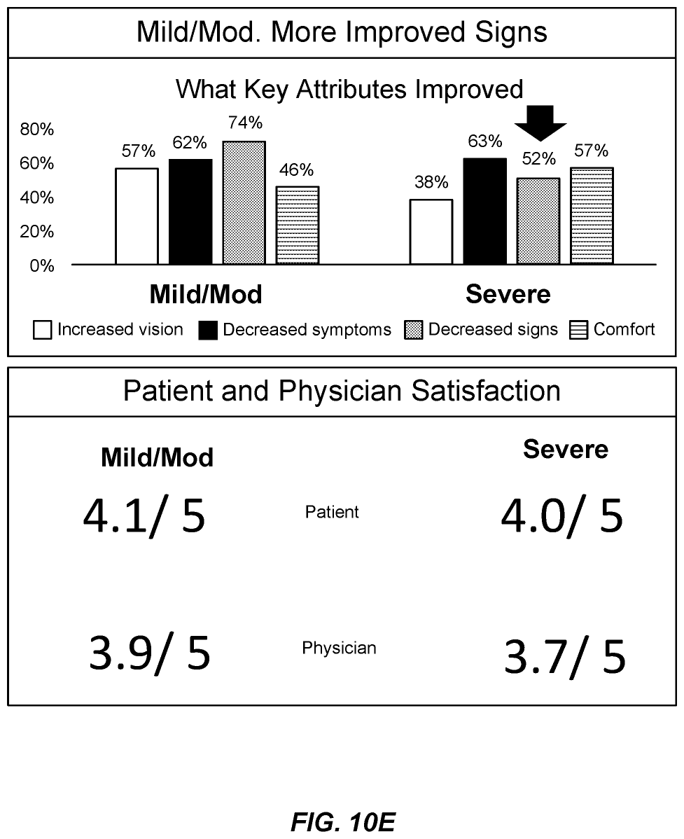

The UC strips were prepared and used similarly to Example 3. The UC strips were evaluated for tolerance, clinical benefits, ease of use, retention, patient satisfaction, and physician satisfaction for mild/moderate and severe indications. Survey data from patients who used the UC strips is seen in FIGS. 10A-10E. For mild/moderate dry eye disease, the UC strips were found to be clinically effective, to be easy to insert, to be tolerable in eye, have good patient/physician satisfaction, and improves all key attributes presented by patients (reduced vision, symptoms, signs) (FIGS. 10A-10B). The UC strips were found to last in the eye for less than a day in 29% cases (FIG. 10A). Similar results were observed for severe indications (FIGS. 10C-10E).

The Example shows that the UC strips were easy to use and clinically effective.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating an ocular surface disease, disorder, and/or wound, comprising:
placing an umbilical cord sheet of about 0.75-2.5 cm by about 0.05-0.75 cm in an inferior fornix, superior fornix, or both of an eye of a subject having an ocular surface disease, disorder, and/or wound.

2. The method of claim 1, further comprising retracting an upper eyelid, lower eyelid, or both of the subject to expose the inferior fornix, the superior fornix, or both prior to placing the umbilical cord sheet.

3. The method of claim 1 or 2, further comprising patching or taping the eye closed.

4. The method of any one of claims 1-3, further comprising administering to the eye a fetal support tissue product selected from the group consisting of: placental amniotic membrane, placenta, chorion, umbilical cord, umbilical cord amniotic membrane, and a combination thereof.

5. The method of any one of claims 1-4, further comprising administering to the eye a therapeutic agent selected from the group consisting of: a steroid, an antibacterial agent, an anti-inflammatory agent, a dry eye treatment agent, an anti-fungal agent, an anti-viral agent, an anti-glaucoma agent, and any combination thereof.

6. The method of claim 4 or 5, wherein the therapeutic agent or the fetal support tissue product is administered to the inferior fornix of the eye, a corneal surface of the eye, and/or tissue surrounding the eye.

7. The method of claim 4 or 5, wherein the therapeutic agent or the fetal support tissue product is administered prior to placing the umbilical cord sheet.

8. The method of any one of claims 1-7, wherein the umbilical cord sheet comprises umbilical cord amniotic membrane and Wharton's jelly.

9. The method of any one of claims 1-8, wherein the umbilical cord sheet is from frozen or previously-frozen umbilical cord.

10. The method of any one of claims 1-9, wherein the umbilical cord sheet does not comprise a cell with metabolic activity, or is substantially free of cells with metabolic activity.

11. The method of any one of claims 1-9, wherein the umbilical cord sheet comprises cells all, substantially all, or most of which are dead.

12. The method of any one of claims 1-9, wherein the umbilical cord sheet is substantially free of living cells.

13. The method of any one of claims 1-12, wherein the umbilical cord sheet is substantially free of red blood cells.

14. The method of any one of claims 1-13, wherein the umbilical cord sheet is substantially free of a vein or an artery.

15. The method of any one of claims 1-14, wherein the umbilical cord sheet is cryopreserved, terminally sterilized, or both.

16. The method of any one of claims 1-15, wherein the umbilical cord sheet is substantially flat.

17. The method of any one of claims 1-16, wherein the umbilical cord sheet is semi-circular.

18. The method of any one of claims 1-16, wherein the umbilical cord sheet is circular.

19. The method of any one of claims 1-16, wherein the umbilical cord sheet is rectangular.

20. The method of any one of claims 1-19, wherein the umbilical cord sheet is about 1.0-2.0 cm by about 0.1-0.5 cm.

21. The method of any one of claims 1-19, wherein the umbilical cord sheet is about 1.5 cm by about 0.3 cm.

22. The method of any one of claims 1-21, wherein the umbilical cord sheet is hydrated.

23. The method of any one of claims 1-22, wherein the umbilical cord sheet promotes nerve regeneration in the eye of the subject.

24. The method of any one of claims 1-23, wherein the umbilical cord sheet is anti-inflammatory, anti-scarring, anti-angiogenic, anti-adhesion, pain-reducing, promotes wound healing, or a combination thereof.

25. The method of any one of claims 1-24, wherein the ocular disease or disorder is selected from the group consisting of dry eye disease, recurrent corneal erosion (RCE), corneal ulcers, herpes simplex keratitis (HSK), recalcitrant punctate keratitis, allergic conjunctivitis, pterygium, infectious diseases, and inflammatory diseases.

26. The method of any one of claims 1-24, wherein the wound is a surgical wound.

27. The method of any one of claims 1-24, wherein the wound is from photorefractive keratectomy (PRK), LASIK, corneal collagen cross-linking (CXL), corneal transplantation, cataract surgery, retinal surgery, or a glaucoma drainage device or filtering bleb.

28. The method of any one of claims 1-24, wherein the wound is from an injury, burn, laceration, incision, or abrasion.

29. A kit for treatment of an ocular surface disease, disorder, or wound in a subject in need thereof comprising:
an umbilical cord sheet; and
a device for placing the umbilical cord sheet onto a corneal surface, into an inferior fornix, a superior fornix, or onto a tissue surrounding an eye of the subject, the device comprising:
a storage unit configured to contain the umbilical cord sheet in a solution; and
a channel operatively coupled to the storage unit, the channel configured such that the umbilical cord sheet passes through the channel from the storage unit and through an opening in the channel, the opening configured to deliver the umbilical cord sheet onto the corneal surface, inferior fornix, the superior fornix, or tissue surrounding the eye.

30. The kit of claim 29, wherein the device further comprises an application member operatively coupled to the channel, wherein the application member is configured to push the umbilical cord sheet through the opening.

31. The kit of any one of claims 29-30, wherein the channel comprises a porous barrier comprising one or more pores, wherein at least one pore of the one or more pores is sized to inhibit the umbilical cord sheet from passing therethrough.

32. The kit of any one of claims 29-31, wherein the umbilical cord sheet does not comprise a cell with metabolic activity, or is substantially free of cells with metabolic activity.

33. The kit of any one of claims 29-32, wherein the umbilical cord sheet comprises cells substantially all of which are dead.

34. The kit of any one of claims 29-33, wherein the umbilical cord sheet is substantially-flattened.

35. The kit of any one of claims 29-34, wherein the umbilical cord sheet is semi-circular.

36. The kit of any one of claims 29-34, wherein the umbilical cord sheet is circular.

37. The kit of any one of claims 29-34, wherein the umbilical cord sheet is rectangular.

38. The kit of any one of claims 29-37, wherein the umbilical cord sheet comprises umbilical cord amniotic membrane and Wharton's jelly.

39. The kit of any one of claims 29-38, wherein the umbilical cord sheet is from frozen or previously-frozen umbilical cord.

40. The kit of any one of claims 29-39, wherein the umbilical cord sheet is hydrated.

41. The kit of any one of claims 29-40, wherein the umbilical cord sheet promotes nerve regeneration in the eye of the subject.

42. The kit of any one of claims 29-41, wherein the umbilical cord sheet is anti-inflammatory, anti-scarring, anti-angiogenic, anti-adhesion, pain-reducing, promotes wound healing, or a combination thereof.

43. The kit of any one of claims 29-42, further comprising a therapeutic agent selected from the group consisting of: a steroid, an antibacterial agent, an anti-inflammatory agent, a dry eye treatment agent, an anti-fungal agent, an anti-viral agent, an anti-glaucoma agent, and any combination thereof.

44. The kit of claim 43, further comprising a fetal support tissue product selected from the group consisting of: placental amniotic membrane, placenta, chorion, umbilical cord, umbilical cord amniotic membrane, and a combination thereof.

45. A method of treating an ocular surface disease, disorder, or wound in a subject in need thereof, comprising:
administering to an inferior fornix, a superior fornix, a corneal surface of an eye of the subject a composition comprising isolated HC-HA/PTX3, and an excipient for controlled-release of the HC-HA/PTX3.

46. The method of claim 45, wherein the excipient for controlled-release comprises a biodegradable polymer.

47. The method of claim 46, wherein the excipient for controlled-release comprises collagen, cellulose, chitosan, PEG, poly(N-isopropylacrylamide), poly(lactic) acid, poly(lactic-co-glycolic) acid, or a combination thereof.

48. The method of any one of claims 45-47, wherein the composition conforms to the inferior fornix, the superior fornix, or the corneal surface.

49. The method of any one of claims 45-48, wherein the composition is transparent, or translucent.

50. The method of any one of claims 45-49, wherein the composition is a solid or semi-solid.

51. The method of claim 50, wherein the composition comprises an opening.

52. The method of claim 50 or 51, wherein the composition is semi-circular.

53. The method of claim 50 or 51, wherein the compositions circular.

54. The method of claim 50 or 51, wherein the composition is rectangular.

55. The method of any one of claims 50-54, wherein the composition is about 1.0-2.0 cm by about 0.1-0.5 cm.

56. The method of any one of claims 50-54, wherein the composition is about 1.5 cm by about 0.3 cm.

57. The method of any one of claims 45-56, further comprising administering to the eye a therapeutic agent selected from the group consisting of: a steroid, an antibacterial agent, an anti-inflammatory agent, a dry eye treatment agent, an anti-fungal agent, an anti-viral agent, an anti-glaucoma agent, and any combination thereof.

58. The method of any one of claims 45-56, further comprising administering to the eye a fetal support tissue product selected from the group consisting of: placental amniotic membrane, placenta, chorion, umbilical cord, umbilical cord amniotic membrane, and a combination thereof.

59. The method of claim 57 or 58, wherein the therapeutic agent or the fetal support tissue is administered to the inferior fornix of the eye, the superior fornix of the eye, a corneal surface of the eye, and/or tissue surrounding the eye.

60. The method of any one of claims 57-59, wherein the therapeutic agent or the fetal support tissue product is administered prior to the composition.

61. A composition, comprising:
isolated HC-HA/PTX3 complex and an excipient for controlled-release, wherein the composition is configured to conforms to a corneal surface or fits in an inferior fornix, a superior fornix, or both of a human eye.

62. The composition of claim 61, wherein the composition is anti-inflammatory, anti-scarring, anti-angiogenic, anti-adhesion, pain-reducing, promotes wound healing, or a combination thereof.

63. The composition of claim 61 or 62, wherein the excipient for controlled-release comprises a biodegradable polymer.

64. The composition of claim 63, wherein the excipient for controlled-release comprises collagen, cellulose, chitosan, PEG, poly(N-isopropylacrylamide), poly(lactic) acid, poly(lactic-co-glycolic) acid, or a combination thereof.

65. The composition of any one of claims 61-64, wherein the composition is transparent, or translucent.

66. The composition of claims 61-65, wherein the composition is a solid or semi-solid.

67. The composition of claim 66, wherein the composition is flat or substantially flat.

68. The composition of claim 66 or 67, wherein the composition comprises an opening.

69. The composition of any one of claims 66-68, wherein the composition is semi-circular.

70. The composition of any one of claims 66-68, wherein the composition is circular.

71. The composition of any one of claims 66-68, wherein the composition is rectangular.

72. The composition of any one of claims 66-71, wherein the composition is about 1.0-2.0 cm by about 0.1-0.5 cm.

73. The composition of any one of claims 66-71, wherein the composition is about 1.5 cm by about 0.3 cm.

74. A kit for treatment of an ocular surface disease, disorder, or wound in a subject comprising:
a composition comprising isolated HC-HA/PTX3 and an excipient for controlled-release of HC-HA/PTX3; and
a device for placing the composition onto a corneal surface, into an inferior fornix, into a superior fornix, or onto a tissue surrounding an eye of the subject, the device comprising:
a storage unit configured to contain the composition in a solution; and
a channel operatively coupled to the storage unit, the channel configured such that the composition passes through the channel from the storage unit and through an opening in the channel, the opening configured to deliver the composition onto the corneal surface, inferior fornix, the superior fornix, or tissue surrounding the eye.

75. The kit of claim 74, wherein the device further comprises an application member operatively coupled to the channel, wherein the application member is configured to push the umbilical cord sheet through the opening so as to be placed onto the corneal surface, inferior fornix, the superior fornix, or tissue surrounding the eye.

76. The kit of claim 74 or 75, wherein the channel comprises a porous barrier comprising one or more pores, wherein at least one pore of the one or more pores is sized to inhibit the umbilical cord sheet from passing therethrough.

77. The kit of any of claims 74-76, wherein the composition is a solid or semi-solid.

78. The kit of claim 77, wherein the composition is semi-circular.

79. The kit of claim 77, wherein the composition is circular.

80. The kit of claim 77, wherein the composition is rectangular.

81. The kit of any one of claims 74-80, wherein the composition promotes nerve regeneration in the eye of the subject.

82. The kit of any one of claims 74-81, further comprising a therapeutic agent selected from the group consisting of: a steroid, an antibacterial agent, an anti-inflammatory agent, a dry eye treatment agent, an anti-fungal agent, an anti-viral agent, an anti-glaucoma agent, and any combination

83. The kit of any one of claims 74-82, further comprising a fetal support tissue product selected from the group consisting of: placental amniotic membrane, placenta, chorion, umbilical cord, umbilical cord amniotic membrane or a combination thereof.

84. A method of treating an ocular surface disease, disorder, and/or wound, comprising:
placing an umbilical cord sheet of about 1.0-2.0 cm by about 0.1-0.5 cm in an inferior fornix only of an eye of a subject having an ocular surface disease, disorder, and/or wound.

85. The method of claim 84, further comprising retracting an upper eyelid, lower eyelid, or both of the subject to expose the inferior fornix prior to placing the umbilical cord sheet.

86. The method of claim 84 or 85, further comprising patching or taping the eye closed.

87. The method of any one of claims 84-86, further comprising administering to the eye a fetal support tissue product selected from the group consisting of: placental amniotic membrane, placenta, chorion, umbilical cord, umbilical cord amniotic membrane, and a combination thereof.

88. The method of any one of claims 84-87, further comprising administering to the eye a therapeutic agent selected from the group consisting of: a steroid, an antibacterial agent, an anti-inflammatory agent, a dry eye treatment agent, an anti-fungal agent, an anti-viral agent, an anti-glaucoma agent, and any combination thereof.

89. The method of any one of claims 84-88, wherein the umbilical cord sheet is about 1.5 cm by about 0.3 cm.

90. A method of treating recurrent corneal erosion (RCE), corneal ulcers, herpes simplex keratitis (HSK), or OSD with recalcitrant punctate keratitis, comprising:
placing an umbilical cord sheet in an inferior fornix, a superior fornix, or both of an eye of a subject having recurrent corneal erosion (RCE), corneal ulcers, herpes simplex keratitis (HSK), or OSD with recalcitrant punctate keratitis.

91. The method of claim 90, further comprising retracting an upper eyelid, lower eyelid, or both of the subject to expose the inferior fornix, the superior fornix, or both prior to placing the umbilical cord sheet.

92. The method of claim 90 or 91, further comprising patching or taping the eye closed.

93. The method of any one of claims 90-92, further comprising administering to the eye a fetal support tissue product selected from the group consisting of: placental amniotic membrane, placenta, chorion, umbilical cord, umbilical cord amniotic membrane, and a combination thereof.

94. The method of any one of claims 90-93, further comprising administering to the eye a therapeutic agent selected from the group consisting of: a steroid, an antibacterial agent, an anti-inflammatory agent, a dry eye treatment agent, an anti-fungal agent, an anti-viral agent, an anti-glaucoma agent, and any combination thereof.

95. The method of claim 93 or 94, wherein the therapeutic agent or the fetal support tissue product is administered to the inferior fornix of the eye, the superior fornix, a corneal surface of the eye, and/or tissue surrounding the eye.

96. The method of claim 93 or 94, wherein the therapeutic agent or the fetal support tissue product is administered prior to placing the umbilical cord sheet.

97. The method of any one of claims 90-96, wherein the umbilical cord sheet is about 1.0-2.0 cm by about 0.1-0.5 cm

98. The method of any one of claims 90-96, wherein the umbilical cord sheet is about 1.5 cm by about 0.3 cm.
